SEQUENCE LISTING (37 sequences)

SEQ ID NO: 1 - DNA, 2027 bp, Zea mays
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacacc

SEQ ID NO: 2 - DNA, 13721 bp, Artificial Sequence
  Vector sequence
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttaccagagctggtcacctttgtccaccaagatggaactgcggcctcgaagcttggcgcgccggccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcggtgacgtggggcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgcgatctgagcttctagaggatccccatcgatgggccccggccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgggcccaggtagaatccgcctgagtcgcaagggtgacttcgcctatattggacgacggcgcgcagagggcgacctctttttgggttacgattgtaggattatcactaaaacaatacatgaacatattcaaatggcaatctctctaaggcattggaaataaatacaaataacagttgggtggagtttttcgacctgagggcgttaaccttctgttaacctaaaagctcttgcccaaacagcagaatcggcgctaattgccagcggcggaacttttccagtttcgcgaaaaatatcgccactggcaaggaatgggtttgagatggcgaagtctgtcctaaaagcagcgcctgtagttgtagggttgacggccttgatggagcgtcatgccgatgccctctcgagccaacttcaagcacatcatcttaaggttttcccgccgcattccgagaagggcattcgaacattcgggccatcggaggcgtccaagctgctcggcgttggcgagtcatatttacggcagaccgcgtctgagatgccagagttgaatgttagcatgagcccgggtggcaggcgaatgttctcaattgaagatatccatgtgattcggaagtatatggatcaggtcggccgcgggaaccggcgctacctgccacatcgtcgaggcggcgagcagcttcaggttatctctgtgatgaatttcaaaggtgggtcgggtaagaccaccaccgccgcgcatctggcgcagtacctcgctatgcgcggatatcgagtcttggccattgatctcgatcctcaagcgagcctttctgcactctttgggagccaaccggagacggacgttggcccgaacgaaacgctctacggcgctataaggtatgatgatgagcaggtggcaatcgaacgagtcgtccgagggacttacattcccgacctccacctgattcctggtaaccttgagctgatggagtttgaacacgatacgccacgcgcgctgatgaaccgcaaagagggcgacacgctcttttatggtcgcatcagccaagtaattgaagatatcgcggataactatgacgtcgtggtcatcgactgccctccccagcttgggtatctcacgctatccgcattgactgcggcgacgtccattcttgtcacggtccatccgcagatgctggatgtgatgtcgatgaaccagtttctggcaatgacatcgaaccttttgcgtgaaatcgagaatgctggcgccaagttcaagtttaattggatgcgctatctgataacccgtttcgaaccgagcgacggaccacagaaccaaatggtaggttatctgcggtcgatttttggcgaaaatgtcctcaattttccgatgcttaaaaccaccgcggtttcggacgctggcctgacaaaccagactctattcgaagtggagcgtggcctgttcacgcgctcgacctatgatcgagccttggaggcgatgaacgccgtcaacgacgagatcgaaacactgatcaaaaaagcatggggtaggcccacatgagccggaagcacatccttggcgtctcaactgacgcccctgagacgtcgcccgccgacaataggacggcaaagaaccgctccatgccgctcctcggcgtaacaaggaaggagcgcgatccggcaacgaagctcacagcgaacattggtaacgcactgcgagagcaaaacgatcgtcttagccgtgccgaagagatcgagcggcgtctcgctgaaggtcaggcagtgatagagttggatgcctcgtcaatagaaccgtctttcgtgcaggatcgtatgcgaggggacattgacgggctccttacttcgatccgggaacaaggacagcaagtcccaatccttgtgcgaccgcatccgagccagccgggccgatatcaggttgccttcggccaccgccggctacgcgccgtttcagaactcggacttccggtcagagcggtcgttcgcgaactgacggacgagcaagtggtcgtagcacagggtcaggaaaacaatgagcgcgaagatcttaccttcatcgaaaaggcgcgcttcgcacatcgcctgaacaggcagttttctcgagagattgtcatcgccgcgatgtcgatcgacaagagcaatttgtccaagatgcttctgctcgttgacgccctcccctctgaactgaccgatgctattggtgccgctcctggtgttggacggccgagttggcaacaacttgccgagctgattgagaaagtttcttcaccggccgacgtggctaaatatgctatgtcggaggaagttcaagcgctgccatcggcagaacgattcaaggcggtgatcgctagtctgaagcccagtcgggttgcgcgtggacttcccgaggtcatggccaccccagacggcaccagaattgcacaggtgacgcagagcaaggccaaactggaaatcacgattgacaggaaggcgacgcccgattttgcgaccttcgtgctcgatcatgtgccagcgctgtatcaagcgtaccacgctgagaaccaacggaaacggggagagtaaaccgcaaaagaaaagagccccctcaacgtcgccgtcgcggaagcccttctgtctctctagcgcgaacagaatcgcatttcctcgaatcctcgtcaagagtttttagcgccgttttggtgagctgatttcctttgcctgctgaaaggtgaaagatgatgcagacaggaagtgtaacgacgccattcgggcggcggccaatgacgcttgcgcttgtgcggcgccagacggcgctggccgatatcaaacaaggcaagacagcggacaagtggaaggtctttagagacgcgtccgcggctatggaactacttggaatccagtccaacagtcttgccgtccttgatgcgctattgagctttcacccggaaacggagttgcgtcaggaggcacagctgatcgtcttcccgtcgaatgctcagcttgcccttcgggcgcatgggatggctggcgcgactttgcgtaggcacatcgccatgctcgtggagtcaggcttgatcgtccggaaggatagcgccaacggaaagcgttacgctcgtaaggatggcgctggtcagatcgagcgcgcgtttggcttcgatttgtctccgcttctcgcgcggtccgaagagctagcgatgatggcacagcaggtgatggccgatcgagcagcattcaggatggccaaagaaagtctgacgatttgccgacgggacgttcggaagctaattacggcagctatggaagagggagcggagggcgactggcaagctgtcgaggaagtctatgtggaacttgtgggtagaattccacgcgccccgacgcttgctgatgtagagtcaattctcgaagagatgtggatgctccaggaagagataatcaaccggttggaaattagagacaattcagaaaataatagcaccaatgctgcccagagcgagcagcacatacagaattcaaaacccgaatccgttaatgaacttgaacctcgctctgaaaaggagcagggcgctaagccgagtgaaatagaccgggcaaggagcgagccgataaaagcgttccccctcgggatgatcctgaaagcatgcccgaccattggcaattatgggccgagcggtgcggttgctagctggcgtgacctcatgtcggctgcggtggtggttcggtctatgctgggggtcagcccgtcggcttaccaagacgcgtgtgaggcaatgggaccggagaatgcggcagcagcgatggcgtgcattttggagcgagcgaacttcatcaattcgcccgggggctatctccgagatctgacacggcggagcgagcttgggaagttttcacttggcccgatgataatggcgctcttgaaggctagcgggcaggggacgttgcggtttggctagaattagcgagtatggagcaggatggtctgtggtcagctgaccacagacctaataggttgaaaacatgagcgttttttggatgatcgacagaccatccgattcccggagtaccaagcgtgctctgatgggagcgataacattactcaacaagcacgaaggccccatgccgatcgttgatcgtgaaggagagcctgctctacatgcggcggtattttgccggccgaggcatgtagtcgcggagcactgcctatttactgccctaggcacaaacgttgactcttggatcgagctggcagacaaagcaataacccacacagaggacgattaatggctgacgaagagatccagaatccgccggacggtactgctgctgccgaagttgagccggctgctcctagaggtagaagagcaaagaaagcaccagccgaaacagcccgcacgggatcgttcaaatccgtgaagccgaaaacccgcggcctcagcaaccgagaaaaactggagaagatcggtcaaatcgaagctcaggtcgctggcggcgcaaccttgaaggacgccgttaagatcgtgggtatttccgttcagacctattatcaatggaagagagctgcggttcaacctgtctcacagaatccggccgtgtctgtttcagttgacgatgaactcggcgagttcatccaactcgaggaggaaaatatgcatggcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactatacgtaaccaactagtgcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagcgtaccgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttag

SEQ ID NO: 3 - DNA, 1552 bp, Zea mays
agtatgcagtggaaaccaacccagattcggacgataggaaagtatcatgtgaatgatttgccaggaaaaggagaggggtaaaaaggggcgaagatttagaagatctaaagcacaagaaccagagattagattgaacaatagggaacatggagcatcctttttttcttcagggaaaaactgaaaatccaaaccatgttgggcaaaaccgagtgggattggaaaccaaaaaacccgagataaagaaactcgagaaaaagcatgaaatcgaaaccaacttcagtaaaacaaaaggaggacagaaaagaaagtcggaaggtataaagaatacattaacattcagtgaaacagcatgctgtcttcttctttttttatgcacaacagagcatacatatataccttcccaggctgaggacttggcggaggagagccgcggagaggatggcggtgcagacggtctggacgggcccgaagacggagacgaacagcgggcccttcctgcccaggcaccacgcttggaacgccatgcacgcgccaaccgcggccccgccgaggacgacgatccccgcgacaagcgtggcgtcgatcctgggcgaccccatgccgaggaacctcccttccaggacgagccgcaggacggcggtgagagaggcacccgtcgcggaggtggcgcagcacatggtgagcagcgcggggaaggcggcgagcgtggcggcctgcaggacggtgacgagcgcgaagacggtgacgccggcgacgaggcagcagcagccgaggatccagtcgtaggaggaagccggaccaaaccgggcaatgcaacctgcagatgcactagacggaggaaacgaggaggaggagaaaacagagcaagagcaggcggagagaagatagagcaaaacacgagtgaggcacagcgtaagcactcggtagaagtctccagaggcgaggtgcgcacaggagaacagatgagtaaagtcagccaaggatccacgatccaacggctacgaatttttggagtgacgtggataggctcaaaggcgccatttccatccggctttatagtattttaaaaaaattcattttcctccctctagtgtgtgcggaggcgtgagcccgtttaacggcgttgagaagtctaacggacaccaaccacaaccaggaaccagcgccggccgcgccgccgagtgaagcagactgcatacggcacggcgcggcatctctctggctgcctctcgagagttccgcccccaccttcccgcggtagcgtggtggtttcgctttccgctgtcggcatccggaagttgcgtgtcagagtggacggagacgaggccgggtcctccagctcctctcaaacgtcacggcaccggcgtccggcagccagcgcggtccttcccaaccactcgttcccaacccatcccccttcctcgcccgccgtcataaatagccagccccatccccagcttctttccccaacctcatcttctctccttttgctcggaacgcacacaccgcccggtctccgatctccgatccccgatcccctcgtcgatccaag

SEQ ID NO: 4 - DNA, 41 bp, Artificial Sequence
  forward primer for P72 promoter
tcgaagcttggcgcgccagtatgcagtggaaaccaacccag

SEQ ID NO: 5 - DNA, 37 bp, Artificial Sequence
  reverse primer for P72 promoter
acaggacggaccatggcttggatcgacgaggggatcg

SEQ ID NO: 6 - DNA, 2386 bp, Zea mays
gtacggcgaccatcctcccccccccccccccccctctctctctgccttctctagatcggcgatccgatccatggttacttggttagggcctgctaactatgttcatgtttgcgttagatccgtgcatggacgcgatctgtacacaccagacgcgttctgattgctagctaactcgccagtacctgggaatcctgggatggctgtagccggccccgcacgcagacgggaccgatttcatgattctctatttttttctttgtttcgttgcctagggtttcgttcgatcgatccgcgttattctttatttccatatattctggtacgatgttgatacggttcgaccgtgctgcttacgttctgtgcgcttgtttgccgggtcatttttaccttgccttttttgtatggtttggttgtggcgatgtggtctggtcgggctgtcgttctagatcggagtagagtgctgtttcaaactgtctagcggatctattagatttggatctgcatgtgtgacatatatcttcgtagttaagatgatgcatctgtatgtgtgacatgcggatctattagatttggatctgtatgtgtgacatatatcttcgtagttgagatgatgcatctgtatgtgtgacatatatcttcgtagttaagattatgcatggaaatatcaatcctttagataaggacgggtatacttgttgctgtgggttttactggtacttcgatagatgcatatacatgatctaacatgcttagatacatgaagtaacatgctgctacggtttaataattcttgagttgatttttactggtacttagatagatgtatatacatgcttagatacatgaagtaacatgctcctacagttcctttaatcattattgagtacctatatattctaataaatcagtatgttttaaattattttgattttactggtacttagatagatgtatatatacatgctcaaacatgcttagatacatgaagtaacatgctgctacggtttagtcattattgagtgcctatatattctaataaatcagtatgttttaaattattttgattttactggtacttagatagatgtatatatacatgctcaaacatgcttagatacatgaagtaatatgctactacggtttaattgttcttgagtacctatatattctaataaatcagtatgttttaaattatttcgattttactggtacttagatagatgtatatatacatgcttagatacatgaagtaacatgctactacggtttaattgttcttgaatacctatatattctaataaatcagtatgttttaaattatttcgattttactggtacttagatagatgtatatatacatgctcgaacatgcttagatacatgaagtaacatgctacatatatattataataaatcagtatgtcttaaattattttgattttactggtacttagatagatgtatatacatgctcaaacatgcttagatacatgaagtaacatgctactacggtttaatcattattgagtacctatatattctaataaatcagtatgttttcaattgttttgattttactggtacttagatatatgtatatatacatgctcgaacatgcttagatacgtgaagtaacatgctactatggttaattgttcttgagtacctatatattctaataaatcagtatgttttaaattatttcgattttactggtacttagatagatgtatatatacatgctcgaacatgcttagatacatgaagtaacatgctactacggtttaatcgttcttgagtacctatatattctaataaatcagtatgtcttaaattatcttgattttactggtacttagatagatgtatatacatgcttagatacatgaagtaacatgctactatgatttaatcgttcttgagtacctatatattctaataaatcagtatgtttttaattattttgattttactggtacttagatagatgtatatatacatgctcgaacatgcttagatacatgaagtaacatgctactacggtttaatcattcttgagtacctatatattctaataaatcagtatgtttttaattattttgatattactggtacttaacatgtttagatacatcatatagcatgcacatgctgctactgtttaatcattcgtgaatacctatatattctaatatatcagtatgtcttctaattattatgattttgatgtacttgtatggtggcatatgctgcagctatgtgtagattttgaatacccagtgtgatgagcatgcatggcgccttcatagttcatatgctgtttatttcctttgagactgttcttttttgttgatagtcaccctgttgtttggtgattcttatgcag

SEQ ID NO: 7 - DNA, 35 bp, Artificial Sequence
  PCR primer
tcgaagcttggcgcgccgtacggcgaccatcctcc

SEQ ID NO: 8 - DNA, 37 bp, Artificial Sequence
  PCR primer
acaggacggaccatggctgcataagaatcaccaaaca

SEQ ID NO: 9 - DNA, 3938 bp, Zea mays
agtatgcagtggaaaccaacccagattcggacgataggaaagtatcatgtgaatgatttgccaggaaaaggagaggggtaaaaaggggcgaagatttagaagatctaaagcacaagaaccagagattagattgaacaatagggaacatggagcatcctttttttcttcagggaaaaactgaaaatccaaaccatgttgggcaaaaccgagtgggattggaaaccaaaaaacccgagataaagaaactcgagaaaaagcatgaaatcgaaaccaacttcagtaaaacaaaaggaggacagaaaagaaagtcggaaggtataaagaatacattaacattcagtgaaacagcatgctgtcttcttctttttttatgcacaacagagcatacatatataccttcccaggctgaggacttggcggaggagagccgcggagaggatggcggtgcagacggtctggacgggcccgaagacggagacgaacagcgggcccttcctgcccaggcaccacgcttggaacgccatgcacgcgccaaccgcggccccgccgaggacgacgatccccgcgacaagcgtggcgtcgatcctgggcgaccccatgccgaggaacctcccttccaggacgagccgcaggacggcggtgagagaggcacccgtcgcggaggtggcgcagcacatggtgagcagcgcggggaaggcggcgagcgtggcggcctgcaggacggtgacgagcgcgaagacggtgacgccggcgacgaggcagcagcagccgaggatccagtcgtaggaggaagccggaccaaaccgggcaatgcaacctgcagatgcactagacggaggaaacgaggaggaggagaaaacagagcaagagcaggcggagagaagatagagcaaaacacgagtgaggcacagcgtaagcactcggtagaagtctccagaggcgaggtgcgcacaggagaacagatgagtaaagtcagccaaggatccacgatccaacggctacgaatttttggagtgacgtggataggctcaaaggcgccatttccatccggctttatagtattttaaaaaaattcattttcctccctctagtgtgtgcggaggcgtgagcccgtttaacggcgttgagaagtctaacggacaccaaccacaaccaggaaccagcgccggccgcgccgccgagtgaagcagactgcatacggcacggcgcggcatctctctggctgcctctcgagagttccgcccccaccttcccgcggtagcgtggtggtttcgctttccgctgtcggcatccggaagttgcgtgtcagagtggacggagacgaggccgggtcctccagctcctctcaaacgtcacggcaccggcgtccggcagccagcgcggtccttcccaaccactcgttcccaacccatcccccttcctcgcccgccgtcataaatagccagccccatccccagcttctttccccaacctcatcttctctccttttgctcggaacgcacacaccgcccggtctccgatctccgatccccgatcccctcgtcgatccaaggtacggcgaccatcctcccccccccccccccccctctctctctgccttctctagatcggcgatccgatccatggttacttggttagggcctgctaactatgttcatgtttgcgttagatccgtgcatggacgcgatctgtacacaccagacgcgttctgattgctagctaactcgccagtacctgggaatcctgggatggctgtagccggccccgcacgcagacgggaccgatttcatgattctctatttttttctttgtttcgttgcctagggtttcgttcgatcgatccgcgttattctttatttccatatattctggtacgatgttgatacggttcgaccgtgctgcttacgttctgtgcgcttgtttgccgggtcatttttaccttgccttttttgtatggtttggttgtggcgatgtggtctggtcgggctgtcgttctagatcggagtagagtgctgtttcaaactgtctagcggatctattagatttggatctgcatgtgtgacatatatcttcgtagttaagatgatgcatctgtatgtgtgacatgcggatctattagatttggatctgtatgtgtgacatatatcttcgtagttgagatgatgcatctgtatgtgtgacatatatcttcgtagttaagattatgcatggaaatatcaatcctttagataaggacgggtatacttgttgctgtgggttttactggtacttcgatagatgcatatacatgatctaacatgcttagatacatgaagtaacatgctgctacggtttaataattcttgagttgatttttactggtacttagatagatgtatatacatgcttagatacatgaagtaacatgctcctacagttcctttaatcattattgagtacctatatattctaataaatcagtatgttttaaattattttgattttactggtacttagatagatgtatatatacatgctcaaacatgcttagatacatgaagtaacatgctgctacggtttagtcattattgagtgcctatatattctaataaatcagtatgttttaaattattttgattttactggtacttagatagatgtatatatacatgctcaaacatgcttagatacatgaagtaatatgctactacggtttaattgttcttgagtacctatatattctaataaatcagtatgttttaaattatttcgattttactggtacttagatagatgtatatatacatgcttagatacatgaagtaacatgctactacggtttaattgttcttgaatacctatatattctaataaatcagtatgttttaaattatttcgattttactggtacttagatagatgtatatatacatgctcgaacatgcttagatacatgaagtaacatgctacatatatattataataaatcagtatgtcttaaattattttgattttactggtacttagatagatgtatatacatgctcaaacatgcttagatacatgaagtaacatgctactacggtttaatcattattgagtacctatatattctaataaatcagtatgttttcaattgttttgattttactggtacttagatatatgtatatatacatgctcgaacatgcttagatacgtgaagtaacatgctactatggttaattgttcttgagtacctatatattctaataaatcagtatgttttaaattatttcgattttactggtacttagatagatgtatatatacatgctcgaacatgcttagatacatgaagtaacatgctactacggtttaatcgttcttgagtacctatatattctaataaatcagtatgtcttaaattatcttgattttactggtacttagatagatgtatatacatgcttagatacatgaagtaacatgctactatgatttaatcgttcttgagtacctatatattctaataaatcagtatgtttttaattattttgattttactggtacttagatagatgtatatatacatgctcgaacatgcttagatacatgaagtaacatgctactacggtttaatcattcttgagtacctatatattctaataaatcagtatgtttttaattattttgatattactggtacttaacatgtttagatacatcatatagcatgcacatgctgctactgtttaatcattcgtgaatacctatatattctaatatatcagtatgtcttctaattattatgattttgatgtacttgtatggtggcatatgctgcagctatgtgtagattttgaatacccagtgtgatgagcatgcatggcgccttcatagttcatatgctgtttatttcctttgagactgttcttttttgttgatagtcaccctgttgtttggtgattcttatgcag

SEQ ID NO: 10 - DNA, 46 bp, Artificial Sequence
  forward primer for P72 promoter plus intron
cggcctcgaagcttggcgcgccagtatgcagtggaaaccaacccag

SEQ ID NO: 11 - DNA, 47 bp, Artificial Sequence
  reverse primer for P72 promoter plus intron
ggtttctacaggacggaccatctgcataagaatcaccaaacaacagg

SEQ ID NO: 12 - DNA, 120 bp, Artificial Sequence
  120bp P72 promoter
cataaatagccagccccatccccagcttctttccccaacctcatcttctctccttttgctcggaacgcacacaccgcccggtctccgatctccgatccccgatcccctcgtcgatccaag

SEQ ID NO: 13 - DNA, 172 bp, Artificial Sequence
  172bp-promoter
cggtccttcccaaccactcgttcccaacccatcccccttcctcgcccgccgtcataaatagccagccccatccccagcttctttccccaacctcatcttctctccttttgctcggaacgcacacaccgcccggtctccgatctccgatccccgatcccctcgtcgatccaag

SEQ ID NO: 14 - DNA, 2558 bp, Artificial Sequence
  Promoter deletion and intron
cggtccttcccaaccactcgttcccaacccatcccccttcctcgcccgccgtcataaatagccagccccatccccagcttctttccccaacctcatcttctctccttttgctcggaacgcacacaccgcccggtctccgatctccgatccccgatcccctcgtcgatccaaggtacggcgaccatcctcccccccccccccccccctctctctctgccttctctagatcggcgatccgatccatggttacttggttagggcctgctaactatgttcatgtttgcgttagatccgtgcatggacgcgatctgtacacaccagacgcgttctgattgctagctaactcgccagtacctgggaatcctgggatggctgtagccggccccgcacgcagacgggaccgatttcatgattctctatttttttctttgtttcgttgcctagggtttcgttcgatcgatccgcgttattctttatttccatatattctggtacgatgttgatacggttcgaccgtgctgcttacgttctgtgcgcttgtttgccgggtcatttttaccttgccttttttgtatggtttggttgtggcgatgtggtctggtcgggctgtcgttctagatcggagtagagtgctgtttcaaactgtctagcggatctattagatttggatctgcatgtgtgacatatatcttcgtagttaagatgatgcatctgtatgtgtgacatgcggatctattagatttggatctgtatgtgtgacatatatcttcgtagttgagatgatgcatctgtatgtgtgacatatatcttcgtagttaagattatgcatggaaatatcaatcctttagataaggacgggtatacttgttgctgtgggttttactggtacttcgatagatgcatatacatgatctaacatgcttagatacatgaagtaacatgctgctacggtttaataattcttgagttgatttttactggtacttagatagatgtatatacatgcttagatacatgaagtaacatgctcctacagttcctttaatcattattgagtacctatatattctaataaatcagtatgttttaaattattttgattttactggtacttagatagatgtatatatacatgctcaaacatgcttagatacatgaagtaacatgctgctacggtttagtcattattgagtgcctatatattctaataaatcagtatgttttaaattattttgattttactggtacttagatagatgtatatatacatgctcaaacatgcttagatacatgaagtaatatgctactacggtttaattgttcttgagtacctatatattctaataaatcagtatgttttaaattatttcgattttactggtacttagatagatgtatatatacatgcttagatacatgaagtaacatgctactacggtttaattgttcttgaatacctatatattctaataaatcagtatgttttaaattatttcgattttactggtacttagatagatgtatatatacatgctcgaacatgcttagatacatgaagtaacatgctacatatatattataataaatcagtatgtcttaaattattttgattttactggtacttagatagatgtatatacatgctcaaacatgcttagatacatgaagtaacatgctactacggtttaatcattattgagtacctatatattctaataaatcagtatgttttcaattgttttgattttactggtacttagatatatgtatatatacatgctcgaacatgcttagatacgtgaagtaacatgctactatggttaattgttcttgagtacctatatattctaataaatcagtatgttttaaattatttcgattttactggtacttagatagatgtatatatacatgctcgaacatgcttagatacatgaagtaacatgctactacggtttaatcgttcttgagtacctatatattctaataaatcagtatgtcttaaattatcttgattttactggtacttagatagatgtatatacatgcttagatacatgaagtaacatgctactatgatttaatcgttcttgagtacctatatattctaataaatcagtatgtttttaattattttgattttactggtacttagatagatgtatatatacatgctcgaacatgcttagatacatgaagtaacatgctactacggtttaatcattcttgagtacctatatattctaataaatcagtatgtttttaattattttgatattactggtacttaacatgtttagatacatcatatagcatgcacatgctgctactgtttaatcattcgtgaatacctatatattctaatatatcagtatgtcttctaattattatgattttgatgtacttgtatggtggcatatgctgcagctatgtgtagattttgaatacccagtgtgatgagcatgcatggcgccttcatagttcatatgctgtttatttcctttgagactgttcttttttgttgatagtcaccctgttgtttggtgattcttatgcag

SEQ ID NO: 15 - DNA, 41 bp, Artificial Sequence
  forward primer
tcgaagcttggcgcgcccggtccttcccaaccactcgttcc

SEQ ID NO: 16 - DNA, 37 bp, Artificial Sequence; identical to SEQ ID NO: 8
  reverse primer

SEQ ID NO: 17 - DNA, 328 bp, Artificial Sequence
  328bp-promoter
gcctctcgagagttccgcccccaccttcccgcggtagcgtggtggtttcgctttccgctgtcggcatccggaagttgcgtgtcagagtggacggagacgaggccgggtcctccagctcctctcaaacgtcacggcaccggcgtccggcagccagcgcggtccttcccaaccactcgttcccaacccatcccccttcctcgcccgccgtcataaatagccagccccatccccagcttctttccccaacctcatcttctctccttttgctcggaacgcacacaccgcccggtctccgatctccgatccccgatcccctcgtcgatccaag

SEQ ID NO: 18 - DNA, 2714 bp, Artificial Sequence
  P72 promoter deletion and intron
gcctctcgagagttccgcccccaccttcccgcggtagcgtggtggtttcgctttccgctgtcggcatccggaagttgcgtgtcagagtggacggagacgaggccgggtcctccagctcctctcaaacgtcacggcaccggcgtccggcagccagcgcggtccttcccaaccactcgttcccaacccatcccccttcctcgcccgccgtcataaatagccagccccatccccagcttctttccccaacctcatcttctctccttttgctcggaacgcacacaccgcccggtctccgatctccgatccccgatcccctcgtcgatccaaggtacggcgaccatcctcccccccccccccccccctctctctctgccttctctagatcggcgatccgatccatggttacttggttagggcctgctaactatgttcatgtttgcgttagatccgtgcatggacgcgatctgtacacaccagacgcgttctgattgctagctaactcgccagtacctgggaatcctgggatggctgtagccggccccgcacgcagacgggaccgatttcatgattctctatttttttctttgtttcgttgcctagggtttcgttcgatcgatccgcgttattctttatttccatatattctggtacgatgttgatacggttcgaccgtgctgcttacgttctgtgcgcttgtttgccgggtcatttttaccttgccttttttgtatggtttggttgtggcgatgtggtctggtcgggctgtcgttctagatcggagtagagtgctgtttcaaactgtctagcggatctattagatttggatctgcatgtgtgacatatatcttcgtagttaagatgatgcatctgtatgtgtgacatgcggatctattagatttggatctgtatgtgtgacatatatcttcgtagttgagatgatgcatctgtatgtgtgacatatatcttcgtagttaagattatgcatggaaatatcaatcctttagataaggacgggtatacttgttgctgtgggttttactggtacttcgatagatgcatatacatgatctaacatgcttagatacatgaagtaacatgctgctacggtttaataattcttgagttgatttttactggtacttagatagatgtatatacatgcttagatacatgaagtaacatgctcctacagttcctttaatcattattgagtacctatatattctaataaatcagtatgttttaaattattttgattttactggtacttagatagatgtatatatacatgctcaaacatgcttagatacatgaagtaacatgctgctacggtttagtcattattgagtgcctatatattctaataaatcagtatgttttaaattattttgattttactggtacttagatagatgtatatatacatgctcaaacatgcttagatacatgaagtaatatgctactacggtttaattgttcttgagtacctatatattctaataaatcagtatgttttaaattatttcgattttactggtacttagatagatgtatatatacatgcttagatacatgaagtaacatgctactacggtttaattgttcttgaatacctatatattctaataaatcagtatgttttaaattatttcgattttactggtacttagatagatgtatatatacatgctcgaacatgcttagatacatgaagtaacatgctacatatatattataataaatcagtatgtcttaaattattttgattttactggtacttagatagatgtatatacatgctcaaacatgcttagatacatgaagtaacatgctactacggtttaatcattattgagtacctatatattctaataaatcagtatgttttcaattgttttgattttactggtacttagatatatgtatatatacatgctcgaacatgcttagatacgtgaagtaacatgctactatggttaattgttcttgagtacctatatattctaataaatcagtatgttttaaattatttcgattttactggtacttagatagatgtatatatacatgctcgaacatgcttagatacatgaagtaacatgctactacggtttaatcgttcttgagtacctatatattctaataaatcagtatgtcttaaattatcttgattttactggtacttagatagatgtatatacatgcttagatacatgaagtaacatgctactatgatttaatcgttcttgagtacctatatattctaataaatcagtatgtttttaattattttgattttactggtacttagatagatgtatatatacatgctcgaacatgcttagatacatgaagtaacatgctactacggtttaatcattcttgagtacctatatattctaataaatcagtatgtttttaattattttgatattactggtacttaacatgtttagatacatcatatagcatgcacatgctgctactgtttaatcattcgtgaatacctatatattctaatatatcagtatgtcttctaattattatgattttgatgtacttgtatggtggcatatgctgcagctatgtgtagattttgaatacccagtgtgatgagcatgcatggcgccttcatagttcatatgctgtttatttcctttgagactgttcttttttgttgatagtcaccctgttgtttggtgattcttatgcag

SEQ ID NO: 19 - DNA, 40 bp, Artificial Sequence
  forward primer
tcgaagcttggcgcgccgcctctcgagagttccgccccca

SEQ ID NO: 20 - DNA, 37 bp, Artificial Sequence; identical to SEQ ID NO: 8
  reverse primer

SEQ ID NO: 21 - DNA, 518 bp, Artificial Sequence
  518bp-promoter
gcgccatttccatccggctttatagtattttaaaaaaattcattttcctccctctagtgtgtgcggaggcgtgagcccgtttaacggcgttgagaagtctaacggacaccaaccacaaccaggaaccagcgccggccgcgccgccgagtgaagcagactgcatacggcacggcgcggcatctctctggctgcctctcgagagttccgcccccaccttcccgcggtagcgtggtggtttcgctttccgctgtcggcatccggaagttgcgtgtcagagtggacggagacgaggccgggtcctccagctcctctcaaacgtcacggcaccggcgtccggcagccagcgcggtccttcccaaccactcgttcccaacccatcccccttcctcgcccgccgtcataaatagccagccccatccccagcttctttccccaacctcatcttctctccttttgctcggaacgcacacaccgcccggtctccgatctccgatccccgatcccctcgtcgatccaag

SEQ ID NO: 22 - DNA, 2904 bp, Artificial Sequence
  P72 promoter deletion and intron
gcgccatttccatccggctttatagtattttaaaaaaattcattttcctccctctagtgtgtgcggaggcgtgagcccgtttaacggcgttgagaagtctaacggacaccaaccacaaccaggaaccagcgccggccgcgccgccgagtgaagcagactgcatacggcacggcgcggcatctctctggctgcctctcgagagttccgcccccaccttcccgcggtagcgtggtggtttcgctttccgctgtcggcatccggaagttgcgtgtcagagtggacggagacgaggccgggtcctccagctcctctcaaacgtcacggcaccggcgtccggcagccagcgcggtccttcccaaccactcgttcccaacccatcccccttcctcgcccgccgtcataaatagccagccccatccccagcttctttccccaacctcatcttctctccttttgctcggaacgcacacaccgcccggtctccgatctccgatccccgatcccctcgtcgatccaaggtacggcgaccatcctcccccccccccccccccctctctctctgccttctctagatcggcgatccgatccatggttacttggttagggcctgctaactatgttcatgtttgcgttagatccgtgcatggacgcgatctgtacacaccagacgcgttctgattgctagctaactcgccagtacctgggaatcctgggatggctgtagccggccccgcacgcagacgggaccgatttcatgattctctatttttttctttgtttcgttgcctagggtttcgttcgatcgatccgcgttattctttatttccatatattctggtacgatgttgatacggttcgaccgtgctgcttacgttctgtgcgcttgtttgccgggtcatttttaccttgccttttttgtatggtttggttgtggcgatgtggtctggtcgggctgtcgttctagatcggagtagagtgctgtttcaaactgtctagcggatctattagatttggatctgcatgtgtgacatatatcttcgtagttaagatgatgcatctgtatgtgtgacatgcggatctattagatttggatctgtatgtgtgacatatatcttcgtagttgagatgatgcatctgtatgtgtgacatatatcttcgtagttaagattatgcatggaaatatcaatcctttagataaggacgggtatacttgttgctgtgggttttactggtacttcgatagatgcatatacatgatctaacatgcttagatacatgaagtaacatgctgctacggtttaataattcttgagttgatttttactggtacttagatagatgtatatacatgcttagatacatgaagtaacatgctcctacagttcctttaatcattattgagtacctatatattctaataaatcagtatgttttaaattattttgattttactggtacttagatagatgtatatatacatgctcaaacatgcttagatacatgaagtaacatgctgctacggtttagtcattattgagtgcctatatattctaataaatcagtatgttttaaattattttgattttactggtacttagatagatgtatatatacatgctcaaacatgcttagatacatgaagtaatatgctactacggtttaattgttcttgagtacctatatattctaataaatcagtatgttttaaattatttcgattttactggtacttagatagatgtatatatacatgcttagatacatgaagtaacatgctactacggtttaattgttcttgaatacctatatattctaataaatcagtatgttttaaattatttcgattttactggtacttagatagatgtatatatacatgctcgaacatgcttagatacatgaagtaacatgctacatatatattataataaatcagtatgtcttaaattattttgattttactggtacttagatagatgtatatacatgctcaaacatgcttagatacatgaagtaacatgctactacggtttaatcattattgagtacctatatattctaataaatcagtatgttttcaattgttttgattttactggtacttagatatatgtatatatacatgctcgaacatgcttagatacgtgaagtaacatgctactatggttaattgttcttgagtacctatatattctaataaatcagtatgttttaaattatttcgattttactggtacttagatagatgtatatatacatgctcgaacatgcttagatacatgaagtaacatgctactacggtttaatcgttcttgagtacctatatattctaataaatcagtatgtcttaaattatcttgattttactggtacttagatagatgtatatacatgcttagatacatgaagtaacatgctactatgatttaatcgttcttgagtacctatatattctaataaatcagtatgtttttaattattttgattttactggtacttagatagatgtatatatacatgctcgaacatgcttagatacatgaagtaacatgctactacggtttaatcattcttgagtacctatatattctaataaatcagtatgtttttaattattttgatattactggtacttaacatgtttagatacatcatatagcatgcacatgctgctactgtttaatcattcgtgaatacctatatattctaatatatcagtatgtcttctaattattatgattttgatgtacttgtatggtggcatatgctgcagctatgtgtagattttgaatacccagtgtgatgagcatgcatggcgccttcatagttcatatgctgtttatttcctttgagactgttcttttttgttgatagtcaccctgttgtttggtgattcttatgcag

SEQ ID NO: 23 - DNA, 38 bp, Artificial Sequence
  forward primer
tcgaagcttggcgcgccgcgccatttccatccggcttt

SEQ ID NO: 24 - DNA, 37 bp, Artificial Sequence; identical to SEQ ID NO: 8
  reverse primer

SEQ ID NO: 25 - DNA, 1036 bp, Artificial Sequence
  1036bp-promoter
aacgccatgcacgcgccaaccgcggccccgccgaggacgacgatccccgcgacaagcgtggcgtcgatcctgggcgaccccatgccgaggaacctcccttccaggacgagccgcaggacggcggtgagagaggcacccgtcgcggaggtggcgcagcacatggtgagcagcgcggggaaggcggcgagcgtggcggcctgcaggacggtgacgagcgcgaagacggtgacgccggcgacgaggcagcagcagccgaggatccagtcgtaggaggaagccggaccaaaccgggcaatgcaacctgcagatgcactagacggaggaaacgaggaggaggagaaaacagagcaagagcaggcggagagaagatagagcaaaacacgagtgaggcacagcgtaagcactcggtagaagtctccagaggcgaggtgcgcacaggagaacagatgagtaaagtcagccaaggatccacgatccaacggctacgaatttttggagtgacgtggataggctcaaaggcgccatttccatccggctttatagtattttaaaaaaattcattttcctccctctagtgtgtgcggaggcgtgagcccgtttaacggcgttgagaagtctaacggacaccaaccacaaccaggaaccagcgccggccgcgccgccgagtgaagcagactgcatacggcacggcgcggcatctctctggctgcctctcgagagttccgcccccaccttcccgcggtagcgtggtggtttcgctttccgctgtcggcatccggaagttgcgtgtcagagtggacggagacgaggccgggtcctccagctcctctcaaacgtcacggcaccggcgtccggcagccagcgcggtccttcccaaccactcgttcccaacccatcccccttcctcgcccgccgtcataaatagccagccccatccccagcttctttccccaacctcatcttctctccttttgctcggaacgcacacaccgcccggtctccgatctccgatccccgatcccctcgtcgatccaag

SEQ ID NO: 26 - DNA, 3422 bp, Artificial Sequence
  P72 promoter deletion and intron
aacgccatgcacgcgccaaccgcggccccgccgaggacgacgatccccgcgacaagcgtggcgtcgatcctgggcgaccccatgccgaggaacctcccttccaggacgagccgcaggacggcggtgagagaggcacccgtcgcggaggtggcgcagcacatggtgagcagcgcggggaaggcggcgagcgtggcggcctgcaggacggtgacgagcgcgaagacggtgacgccggcgacgaggcagcagcagccgaggatccagtcgtaggaggaagccggaccaaaccgggcaatgcaacctgcagatgcactagacggaggaaacgaggaggaggagaaaacagagcaagagcaggcggagagaagatagagcaaaacacgagtgaggcacagcgtaagcactcggtagaagtctccagaggcgaggtgcgcacaggagaacagatgagtaaagtcagccaaggatccacgatccaacggctacgaatttttggagtgacgtggataggctcaaaggcgccatttccatccggctttatagtattttaaaaaaattcattttcctccctctagtgtgtgcggaggcgtgagcccgtttaacggcgttgagaagtctaacggacaccaaccacaaccaggaaccagcgccggccgcgccgccgagtgaagcagactgcatacggcacggcgcggcatctctctggctgcctctcgagagttccgcccccaccttcccgcggtagcgtggtggtttcgctttccgctgtcggcatccggaagttgcgtgtcagagtggacggagacgaggccgggtcctccagctcctctcaaacgtcacggcaccggcgtccggcagccagcgcggtccttcccaaccactcgttcccaacccatcccccttcctcgcccgccgtcataaatagccagccccatccccagcttctttccccaacctcatcttctctccttttgctcggaacgcacacaccgcccggtctccgatctccgatccccgatcccctcgtcgatccaaggtacggcgaccatcctcccccccccccccccccctctctctctgccttctctagatcggcgatccgatccatggttacttggttagggcctgctaactatgttcatgtttgcgttagatccgtgcatggacgcgatctgtacacaccagacgcgttctgattgctagctaactcgccagtacctgggaatcctgggatggctgtagccggccccgcacgcagacgggaccgatttcatgattctctatttttttctttgtttcgttgcctagggtttcgttcgatcgatccgcgttattctttatttccatatattctggtacgatgttgatacggttcgaccgtgctgcttacgttctgtgcgcttgtttgccgggtcatttttaccttgccttttttgtatggtttggttgtggcgatgtggtctggtcgggctgtcgttctagatcggagtagagtgctgtttcaaactgtctagcggatctattagatttggatctgcatgtgtgacatatatcttcgtagttaagatgatgcatctgtatgtgtgacatgcggatctattagatttggatctgtatgtgtgacatatatcttcgtagttgagatgatgcatctgtatgtgtgacatatatcttcgtagttaagattatgcatggaaatatcaatcctttagataaggacgggtatacttgttgctgtgggttttactggtacttcgatagatgcatatacatgatctaacatgcttagatacatgaagtaacatgctgctacggtttaataattcttgagttgatttttactggtacttagatagatgtatatacatgcttagatacatgaagtaacatgctcctacagttcctttaatcattattgagtacctatatattctaataaatcagtatgttttaaattattttgattttactggtacttagatagatgtatatatacatgctcaaacatgcttagatacatgaagtaacatgctgctacggtttagtcattattgagtgcctatatattctaataaatcagtatgttttaaattattttgattttactggtacttagatagatgtatatatacatgctcaaacatgcttagatacatgaagtaatatgctactacggtttaattgttcttgagtacctatatattctaataaatcagtatgttttaaattatttcgattttactggtacttagatagatgtatatatacatgcttagatacatgaagtaacatgctactacggtttaattgttcttgaatacctatatattctaataaatcagtatgttttaaattatttcgattttactggtacttagatagatgtatatatacatgctcgaacatgcttagatacatgaagtaacatgctacatatatattataataaatcagtatgtcttaaattattttgattttactggtacttagatagatgtatatacatgctcaaacatgcttagatacatgaagtaacatgctactacggtttaatcattattgagtacctatatattctaataaatcagtatgttttcaattgttttgattttactggtacttagatatatgtatatatacatgctcgaacatgcttagatacgtgaagtaacatgctactatggttaattgttcttgagtacctatatattctaataaatcagtatgttttaaattatttcgattttactggtacttagatagatgtatatatacatgctcgaacatgcttagatacatgaagtaacatgctactacggtttaatcgttcttgagtacctatatattctaataaatcagtatgtcttaaattatcttgattttactggtacttagatagatgtatatacatgcttagatacatgaagtaacatgctactatgatttaatcgttcttgagtacctatatattctaataaatcagtatgtttttaattattttgattttactggtacttagatagatgtatatatacatgctcgaacatgcttagatacatgaagtaacatgctactacggtttaatcattcttgagtacctatatattctaataaatcagtatgtttttaattattttgatattactggtacttaacatgtttagatacatcatatagcatgcacatgctgctactgtttaatcattcgtgaatacctatatattctaatatatcagtatgtcttctaattattatgattttgatgtacttgtatggtggcatatgctgcagctatgtgtagattttgaatacccagtgtgatgagcatgcatggcgccttcatagttcatatgctgtttatttcctttgagactgttcttttttgttgatagtcaccctgttgtttggtgattcttatgcag

SEQ ID NO: 27 - DNA, 38 bp, Artificial Sequence
  forward primer
tcgaagcttggcgcgccaacgccatgcacgcgccaacc

SEQ ID NO: 28 - DNA, 37 bp, Artificial Sequence; identical to SEQ ID NO: 8
  reverse primer

SEQ ID NO: 29 - DNA, 21 bp, Artificial Sequence
  GUS fwd primer
cttacgtggcaaaggattcga

SEQ ID NO: 30 - DNA, 20 bp, Artificial Sequence
  GUS rev primer
gccccaatccagtccattaa

SEQ ID NO: 31 - DNA, 21 bp, Artificial Sequence
  GR5 fwd primer
ggcagtttggttgatgctcat

SEQ ID NO: 32 - DNA, 26 bp, Artificial Sequence
  GR5 rev primer
tgctgtatatctttgctttgaaccat

SEQ ID NO: 33 - DNA, 20 bp, Artificial Sequence
  ADH fwd primer
caagtcgcggttttcaatca

SEQ ID NO: 34 - DNA, 21 bp, Artificial Sequence
  ADH rev primer
tgaaggtggaagtcccaacaa

SEQ ID NO: 35 - DNA, 23 bp, Artificial Sequence
  GUS probe sequence
aacgtgctgatggtgcacgacca

SEQ ID NO: 36 - DNA, 17 bp, Artificial Sequence
  GR5 probe sequence
ttgaagtcacaaagcca

SEQ ID NO: 37 - DNA, 19 bp, Artificial Sequence
  ADH probe sequence
tgggaagcctatctaccac

(19) United States
(12) Patent Application Publication
(10) Pub. No.: US 2013/0254932 A1
(43) Pub. Date: Sep. 26, 2013

(54) PLANT GENE EXPRESSION MODULATORY SEQUENCES FROM MAIZE

(71) Applicants: Ajit Nott, Secunderabad (IN); David A. Selinger, Johnston, IA (US); Venkata S. Tavva, Hyderabad (IN)

(72) Inventors: Ajit Nott, Secunderabad (IN); David A. Selinger, Johnston, IA (US); Venkata S. Tavva, Hyderabad (IN)

(73) Assignee: E.I. DU PONT DE NUMOURS AND COMPANY, WILIMINGTON, DE (US)

(21) Appl. No.: 13/990,918

(22) PCT Filed: Dec. 21, 2011
(86) PCT No.: PCT/US11/66633

§ 371 Date: May 31, 2013

Related U.S. Application Data
(60) Provisional application No. 61/466,480, filed on Mar. 23, 2011.

(30) Foreign Application Priority Data
Dec. 21, 2010 (IN) 3060/DELNP/2010

(51) Int. Cl. C12N 15/82

(52) U.S. Cl. CPC C12N 15/82
USPC 800/278; 435/320.1; 800/298

(57) ABSTRACT

The invention relates to gene expression regulatory sequences from maize, specifically to a promoter sequence and an intron sequence, that are useful for expressing transgenes in transgenic plants. The invention further discloses compositions, polynucleotide constructs, transformed host cells, transgenic plants and seeds containing the recombinant construct with the promoter and intron sequences, and methods for preparing and using the same.

### CROSS REFERENCE TO RELATED APPLICATIONS

This application claims the benefit of India Patent Application No. 3060/DELNP/2010, filed Dec. 21, 2010, and U.S. Provisional Application No. 61/466,480, filed Mar. 23, 2011; the entire content of each is herein incorporated by reference.

### FIELD OF THE INVENTION

The present invention relates to the field of plant molecular biology and plant genetic engineering. More specifically, it relates to compositions and methods of use of regulatory sequences such as promoter and intron sequences to regulate gene expression in plants.

### BACKGROUND

Recent advances in plant genetic engineering have opened new doors to engineer plants to have improved characteristics or traits. These transgenic plants characteristically have recombinant DNA constructs in their genome that have a polynucleotide of interest operably linked to at least one regulatory region, e.g., a promoter that allows expression of the transgene. The expression level of the polynucleotide of interest can also be modulated by other regulatory elements such as introns and enhancers. Introns have been reported to affect the levels of gene expression (Intron Mediated Enhancement of gene expression (Lu et al., Mol Genet Genomics (2008) 279:563-572).

Promoters can be strong or weak promoters, or can be constitutive, or might be regulated in a spatiotemporal or inducible manner. Thus, promoters allow transgene expression to be regulated, restricted and fine-tuned, allowing more precise control over the manner in which the transgene, and hence the phenotype conferred by it is expressed. Plant genetic engineering has advanced to introducing multiple traits into commercially important plants, also known as gene stacking. This is accomplished by multigene transformation, where multiple genes are transferred to create a transgenic plant that might express a complex phenotype, or multiple phenotypes. But it is important to modulate or control the expression of each transgene optimally, and the regulatory elements need to be diverse, to avoid introducing into the same transgenic plant repetitive sequences, which has been correlated with undesirable negative effects on transgene expression and stability (Peremarti et al (2010) Plant Mol Biol 73:363-378; Mette et al (1999) EMBO J 18:241-248; Mette et al (2000) EMBO J 19:5194-5201; Mourrain et al (2007) Planta 225:365-379, U.S. Pat. No. 7,632,982, U.S. Pat. No. 7,491,813, U.S. Pat. No. 7,674,950, PCT Application No. PCT/US2009/046968). Therefore it is important to discover and characterize novel regulatory elements that can be used to express heterologous nucleic acids in important crop species. Diverse promoters with desired expression profiles can be used to control the expression of each transgene optimally.

### SUMMARY

The present invention discloses novel regulatory sequences from maize that can be used for regulating gene expression of heterologous polynucleotides in transgenic plants. It discloses a maize promoter and a maize intron sequence that can be used to regulate plant gene expression of heterologous polynucleotides.

One embodiment of this invention is a recombinant DNA construct comprising a promoter functional in a plant cell operably linked to an isolated polynucleotide wherein the promoter comprises a nucleic acid sequence selected from the group consisting of: (a) the nucleic acid sequence of SEQ ID NO: 3, (b) a nucleic acid sequence with at least 95% sequence identity to the nucleic acid sequence of SEQ ID NO: 3, and (c) a nucleic acid sequence comprising a functional fragment of (a) or (b). In a related embodiment, the promoter is a constitutive promoter.

In another embodiment, the recombinant construct may comprise an intron operably linked to both a promoter and an isolated polynucleotide, wherein the intron comprises a nucleic acid sequence with at least 95% identity to the nucleic acid sequence of SEQ ID NO: 6. The intron may further comprise the nucleic acid sequence of SEQ ID NO: 6. In a related embodiment, the expression of the isolated polynucleotide that is operably linked to both an intron and a promoter is enhanced, when compared to a control recombinant DNA construct comprising the promoter operably linked to the isolated polynucleotide, wherein neither are operably linked to the intron.

Another embodiment of this invention is a method for modulating expression of an isolated polynucleotide in a plant comprising the steps of: (a) introducing into a regenerable plant cell a recombinant DNA construct comprising a promoter functional in a plant cell operably linked to an isolated polynucleotide wherein the promoter comprises a nucleic acid sequence selected from the group consisting of: (i) the nucleic acid sequence of SEQ ID NO: 3, (ii) a nucleic acid sequence with at least 95% sequence identity to the nucleic acid sequence of SEQ ID NO: 3, and (iii) a nucleic acid sequence comprising a functional fragment of (i) or (ii); (b) regenerating a transgenic plant from the regenerable plant cell after step (a) wherein the transgenic plant comprises the recombinant DNA construct; and (c) obtaining a progeny plant derived from the transgenic plant of step (b), wherein said progeny plant comprises the recombinant DNA construct, and exhibits expression of the polynucleotide.

Another embodiment of this invention is a method for modulating expression of an isolated polynucleotide in a plant comprising the steps of: (a) introducing into a regenerable plant cell a recombinant DNA construct comprising an intron sequence operably linked to both a promoter and an isolated polynucleotide wherein the intron sequence exhibits at least 95% sequence identity to SEQ ID NO: 6; (b) regenerating a transgenic plant from the regenerable plant cell after step (a) wherein the transgenic plant comprises the recombinant DNA construct; and (c) obtaining a progeny plant derived from the transgenic plant of step (b), wherein said progeny plant comprises the recombinant DNA construct and exhibits enhanced transgene expression when compared to a plant comprising a control recombinant DNA construct comprising the promoter operably linked to the isolated polynucleotide, wherein neither are operably linked to the intron.

Another embodiment of this invention is the method for modulating expression of an isolated polynucleotide in a plant comprising the steps of: (a) introducing into a regenerable plant cell a recombinant DNA construct comprising a promoter functional in a plant cell operably linked to both an intron and an isolated polynucleotide, wherein the promoter comprises the nucleic acid sequence of SEQ ID NO: 3, and wherein the intron comprises the nucleic acid sequence of SEQ ID NO: 6; (b) regenerating a transgenic plant from the regenerable plant cell after step (a) wherein the transgenic plant comprises the recombinant DNA construct; and (c) obtaining a progeny plant derived from the transgenic plant of step (b), wherein said progeny plant comprises the recombinant DNA construct and exhibits expression of the polynucleotide.

An embodiment of this invention is a functional fragment of SEQ ID NO: 3, that comprises at least 50, 100, 200, 300, 400, 500, 1000 or 1500 contiguous nucleotides from the 3′ end of the polynucleotide sequence of SEQ ID NO: 3.

One embodiment of this invention is a functional fragment of SEQ ID NO: 3, wherein the fragment comprises 120 bp (SEQ ID NO: 12), 172 bp (SEQ ID NO: 13), 328 bp (SEQ ID NO: 17), 518 bp (SEQ ID NO: 21) or 1036 bp (SEQ ID NO: 25) of the 3′ end of SEQ ID NO: 3.

Another embodiment of this invention is a recombinant construct comprising a functional fragment of SEQ ID NO: 3 operably linked to an isolated polynucleotide, wherein the functional fragment comprises a nucleotide sequence selected from the group consisting of: (a) the nucleic acid sequence of SEQ ID NOS: 12, 13, 17, 21 or 25; and (b) a nucleic acid sequence with at least 95% sequence identity to the nucleic acid sequence of SEQ ID NOS: 12, 13, 17, 21 or 25.

Another embodiment of this invention is a recombinant construct comprising a functional fragment of SEQ ID NO: 3 operably linked to both an isolated polynucleotide and an intron, wherein the functional fragment comprises a nucleotide sequence selected from the group consisting of: (a) the nucleic acid sequence of SEQ ID NOS: 12, 13, 17, 21 or 25; and (b) a nucleic acid sequence with at least 95% sequence identity to the nucleic acid sequence of SEQ ID NOS: 12, 13, 17, 21 or 25. Furthermore, the intron may comprise the nucleic acid sequence of SEQ ID NO: 6.

Another embodiment of this invention is a method for modulating expression of an isolated polynucleotide in a plant comprising the steps of: (a) introducing into a regenerable plant cell a recombinant DNA construct comprising a functional fragment of SEQ ID NO: 3 operably linked to an isolated polynucleotide and intron, wherein the functional fragment comprises a nucleotide sequence selected from the group consisting of: (i) the nucleic acid sequence of SEQ ID NOS: 12, 13, 17, 21 or 25; and (ii) a nucleic acid sequence with at least 95% sequence identity to the nucleic acid sequence of SEQ ID NOS: 12, 13, 17, 21 or 25; (b) regenerating a transgenic plant from the regenerable plant cell after step (a), wherein the transgenic plant comprises the recombinant DNA construct; and (c) obtaining a progeny plant derived from the transgenic plant of step (b), wherein said progeny plant comprises the recombinant DNA construct and exhibits expression of the polynucleotide. Furthermore, the intron may comprise the sequence of SEQ ID NO: 6.

Another embodiment of this invention is a method for modulating expression of an isolated polynucleotide in a plant comprising the steps of: (a) introducing into a regenerable plant cell a recombinant DNA construct comprising a functional fragment of SEQ ID NO: 3 operably linked to an isolated polynucleotide, wherein the functional fragment comprises a nucleotide sequence selected from the group consisting of: (i) the nucleic acid sequence of SEQ ID NOS: 12, 13, 17, 21 or 25; and (ii) a nucleic acid sequence with at least 95% sequence identity to the nucleic acid sequence of SEQ ID NOS: 12, 13, 17, 21 or 25; (b) regenerating a transgenic plant from the regenerable plant cell after step (a) wherein the transgenic plant comprises the recombinant DNA construct; and (c) obtaining a progeny plant derived from the transgenic plant of step (b), wherein said progeny plant comprises the recombinant DNA construct and exhibits expression of the polynucleotide.

In another embodiment, the compositions and methods of the present invention can be used in dicots or monocots. In particular, the compositions and methods of the present invention can be used in monocotyledenous plants.

In another embodiment, the invention includes transformed plant cells, tissues, plants, and seeds. The invention encompasses regenerated, mature and fertile transgenic plants, transgenic seeds produced therefrom, T1 and subsequent generations. The transgenic plant cells, tissues, plants, and seeds may comprise at least one recombinant DNA construct of interest.

### BRIEF DESCRIPTION OF THE DRAWINGS AND SEQUENCE LISTING

The invention can be more fully understood from the following detailed description and the accompanying drawings and Sequence Listing which form a part of this application.

FIG. 1 shows the map of PHP31993 vector used for testing promoters. The “GUSINT” region of vector PHP31993 designates a β-glucuronidase coding region that has been interrupted with an intron in order to prevent GUS expression in bacteria. The precursor vector PHP31993 was used to create two expression vectors, PHP39158 and PHP38694, in which either the P72 promoter with P72 intron (PHP39158) or the Zm-Ubi promoter with Zm-Ubi intron (PHP38694) was cloned between AscI and NcoI restriction sites. The AcsI and NcoI sites are at the 5′ end of the GUSINT region.

FIG. 2A shows GUS histochemical staining in maize embryos infected with Agrobacterium transformed with PHP39158 construct. The non-transgenic control is labeled as NTC.

FIG. 2B shows quantitative analysis of GUS reporter gene expression in maize embryos infected with transformed Agrobacterium carrying the constructs to be tested. The respective constructs are PHP38694 with Zm-Ubi promoter with Zm-Ubi intron cloned in AscI and NcoI sites and PHP39158 that has P72 promoter with P72 intron to be tested.

FIG. 3A shows GUS histochemical staining in 8 independent transgenic maize callus events transformed with PHP39158, expressing GUS driven by P72 promoter and P72 intron.

FIG. 3B shows quantitative data for GUS protein expression in leaves and pollen tissue from transgenic maize plants transformed with PHP39158, expressing GUS gene driven by P72 promoter and P72 intron and from transgenic maize plants transformed with PHP38694, expressing GUS driven by maize Ubi promoter and Ubi intron. Data depicts average of 3 single copy events for P72 and one single copy event for the maize Ubi promoter control.

FIG. 4A shows GUS histochemical staining of 7 independent transgenic rice callus events expressing GUS reporter gene driven by P72 promoter and P72 intron (PHP39158).

FIG. 4B shows four non-transgenic control calli stained for GUS expression.

FIG. 5A shows histochemical (GUS) data from leaves, stem, roots, tassel, pollen and silk collected from—PHP39158 T1 corn events. Representative images are shown for each tissue analyzed.

FIG. 5B shows histochemical (GUS) data from immature ears collected from PHP39158 T1 corn events.

FIG. 6 shows MUG data from T1 corn events transformed with PHP39158 construct. Data represents the average of 5 independent single copy events±SE.

FIG. 7A-7E show the histochemical data from 1-month-old rice plant for the following tissues: leaf (FIG. 7A), stem (FIG. 7B), boot leaf (FIG. 7C), panicle (FIG. 7D), and anthers (FIG. 7E) collected from stable T0 transgenic events transformed with PHP39158 construct.

FIG. 8 shows MUG data from stable transgenic T0 rice lines transformed with PHP39158 and PHP38694 constructs. Data represents the average of 6 independent single copy events±SE.

SEQ ID NO: 1 is the sequence of Zm-Ubi promoter and intron sequence used as a control for testing promoter activity.

SEQ ID NO: 2 is the sequence of the vector, PHP31993, used for testing promoters.

SEQ ID NO: 3 is the sequence of the P72 promoter.

SEQ ID NO: 4 and 5 are the sequences of the forward and reverse primers, respectively, used for amplifying P72 promoter.

SEQ ID NO: 6 is the sequence of the P72 intron.

SEQ ID NOS: 7 and 8 are the sequences of the forward and reverse primers, respectively, used for amplifying SEQ ID NO: 6.

SEQ ID NO: 9 is the sequence of P72 promoter and P72 intron.

SEQ ID NOS: 10 and 11 are the sequences of the forward and reverse primers, respectively, used for amplifying SEQ ID NO: 9.

SEQ ID NO: 12 is the sequence of a 120-bp P72 promoter fragment.

SEQ ID NO: 13 is the sequence of a 172-bp P72 promoter fragment.

SEQ ID NO: 14 is the sequence of a 172-bp P72 promoter fragment with P72 intron.

SEQ ID NOS: 15 and 16 are the sequences of the forward and reverse primers, respectively, used for amplifying SEQ ID NO: 14.

SEQ ID NO: 17 is the sequence of a 328-bp P72 promoter fragment.

SEQ ID NO: 18 is the sequence of a 328-bp P72 promoter fragment with P72 intron.

SEQ ID NOS: 19 and 20 are the sequences of the forward and reverse primers, respectively, used for amplifying SEQ ID NO: 18.

SEQ ID NO: 21 is the sequence of a 518-bp P72 promoter fragment.

SEQ ID NO: 22 is the sequence of a 518-bp P72 promoter fragment with P72 intron.

SEQ ID NOS: 23 and 24 are the sequences of the forward and reverse primers, respectively, used for amplifying SEQ ID NO: 22.

SEQ ID NO: 25 is the sequence of a 1036-bp P72 promoter fragment.

SEQ ID NO: 26 is the sequence of a 1036-bp P72 promoter fragment with P72 intron.

SEQ ID NOS: 27 and 28 are the sequences of the forward and reverse primers, respectively, used for amplifying SEQ ID NO: 26.

SEQ ID NOS: 29 and 30 are the sequences of the GUS fwd and reverse primers.

SEQ ID NOS: 31 and 32 are the sequences of the GR5 fwd and reverse primers.

SEQ ID NOS: 33 and 34 are the sequences of the ADH fwd and reverse primers.

SEQ ID NO: 35, 36 and 37 are the probe sequences for GUS, GR5 and ADH respectively.

The sequence descriptions and Sequence Listing attached hereto comply with the rules governing nucleotide and/or amino acid sequence disclosures in patent applications as set forth in 37 C.F.R. §1.821-1.825.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

The present invention discloses novel regulatory sequences from maize that can be used for regulating gene expression of heterologous polynucleotides in transgenic plants. It discloses a maize promoter and a maize intron sequence that can be used to regulate plant gene expression of heterologous polynucleotides.

The terms “monocot” and “monocotyledonous plant” are used interchangeably herein. A monocot of the current invention includes the Gramineae.

The terms “dicot” and “dicotyledonous plant” are used interchangeably herein. A dicot of the current invention includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Agronomic characteristic” is a measurable parameter including but not limited to, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell.

“cDNA” refers to a DNA that is complementary to and synthesized from a mRNA template using the enzyme reverse transcriptase. The cDNA can be single-stranded or converted into the double-stranded form using the Klenow fragment of DNA polymerase I.

“Coding region” refers to the portion of a messenger RNA (or the corresponding portion of another nucleic acid molecule such as a DNA molecule) which encodes a protein or polypeptide. “Non-coding region” refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Mature” protein refers to a post-translationally processed polypeptide; i.e., one from which any pre- or pro-peptides present in the primary translation product have been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

The terms “entry clone” and “entry vector” are used interchangeably herein.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

As will be evident to one of skill in the art, any isolated polynucleotide of interest can be operably linked to the regulatory sequences described in the current invention. Examples of polynucleotides of interest that can be operably linked to the regulatory elements described in this invention include, but are not limited to, polynucleotides comprising other regulatory elements such as introns, enhancers, polyadenylation signals, translation leader sequences, protein coding regions such as disease and insect resistance genes, genes conferring nutritional value, genes conferring yield and heterosis increase, genes that confer male and/or female sterility, antifungal, antibacterial or antiviral genes, and the like. Likewise, the promoter and intron sequences described in the current invention can be used to modulate the expression of any nucleic acid to control gene expression. Examples of nucleic acids that could be used to control gene expression include, but are not limited to, antisense oligonucleotides, suppression DNA constructs, or nucleic acids encoding transcription factors.

The promoter described in the current invention can be operably linked to other regulatory sequences. Examples of such regulatory sequences include, but are not limited to, introns, terminators, enhancers, polyadenylation signal sequences, untranslated leader sequences. The promoter sequence described in the present invention can be operably linked to the intronic sequences described herein, but can also be operably linked to other intronic sequences. Other introns are known in art that can enhance gene expression, examples of such introns include, but are not limited to, first intron from Adh1 gene, first intron from Shrunken-1 gene, Callis et al., Genes Dev. 1987 1:1183-1200, Mascarenkas et al., Plant Mol. Biol., 1990, 15: 913-920).

Regulatory Sequences:

A recombinant DNA construct (including a suppression DNA construct) of the present invention may comprise at least one regulatory sequence. In an embodiment of the present invention, the regulatory sequences disclosed herein can be operably linked to any other regulatory sequence.

“Regulatory sequences” or “regulatory elements” are used interchangeably and refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably to refer to a promoter that is expressed predominantly but not necessarily exclusively in one tissue or organ, but that may also be expressed in one specific cell.

“Developmentally regulated promoter” refers to a promoter whose activity is determined by developmental events.

Promoters that cause a gene to be expressed in most cell types at most times are commonly referred to as “constitutive promoters”.

Inducible promoters selectively express an operably linked DNA sequence in response to the presence of an endogenous or exogenous stimulus, for example by chemical compounds (chemical inducers) or in response to environmental, hormonal, chemical, and/or developmental signals. Examples of inducible or regulated promoters include, but are not limited to, promoters regulated by light, heat, stress, flooding or drought, pathogens, phytohormones, wounding, or chemicals such as ethanol, jasmonate, salicylic acid, or safeners.

A minimal or basal promoter is a polynucleotide molecule that is capable of recruiting and binding the basal transcription machinery. One example of basal transcription machinery in eukaryotic cells is the RNA polymerase II complex and its accessory proteins.

Plant RNA polymerase II promoters, like those of other higher eukaryotes, are comprised of several distinct “cis-acting transcriptional regulatory elements,” or simply “cis-elements,” each of which appears to confer a different aspect of the overall control of gene expression. Examples of such cis-acting elements include, but are not limited to, such as TATA box and CCAAT or AGGA box. The promoter can roughly be divided in two parts: a proximal part, referred to as the core, and a distal part. The proximal part is believed to be responsible for correctly assembling the RNA polymerase II complex at the right position and for directing a basal level of transcription, and is also referred to as “minimal promoter” or “basal promoter”. The distal part of the promoter is believed to contain those elements that regulate the spatio-temporal expression. In addition to the proximal and distal parts, other regulatory regions have also been described, that contain enhancer and/or repressors elements The latter elements can be found from a few kilobase pairs upstream from the transcription start site, in the introns, or even at the 3′ side of the genes they regulate (Rombauts, S. et al. (2003) Plant Physiology 132:1162-1176, Nikolov and Burley, (1997) Proc Natl Acad Sci USA 94: 15-22), Tjian and Maniatis (1994) Cell 77: 5-8; Fessele et al., 2002 Trends Genet. 18: 60-63, Messing et al., (1983) Genetic Engineering of Plants: an Agricultural Perspective, Plenum Press, NY, pp 211-227).

When operably linked to a heterologous polynucleotide sequence, a promoter controls the transcription of the linked polynucleotide sequence.

In an embodiment of the present invention, the “cis-acting transcriptional regulatory elements” from the promoter sequence disclosed herein can be operably linked to “cis-acting transcriptional regulatory elements” from any heterologous promoter. Such a chimeric promoter molecule can be engineered to have desired regulatory properties. In an embodiment of this invention a fragment of the disclosed promoter sequence that can act either as a cis-regulatory sequence or a distal-regulatory sequence or as an enhancer sequence or a repressor sequence, may be combined with either a cis-regulatory or a distal regulatory or an enhancer sequence or a repressor sequence or any combination of any of these from a heterologous promoter sequence.

In a related embodiment, a cis-element of the disclosed promoter may confer a particular specificity such as conferring enhanced expression of operably linked polynucleotide molecules in certain tissues and therefore is also capable of regulating transcription of operably linked polynucleotide molecules. Consequently, any fragments, portions, or regions of the promoter comprising the polynucleotide sequence shown in SEQ ID NO: 3 can be used as regulatory polynucleotide molecules.

Promoter fragments that comprise regulatory elements can be added, for example, fused to the 5′ end of, or inserted within, another promoter having its own partial or complete regulatory sequences (Fluhr et al., Science 232:1106-1112, 1986; Ellis et al., EMBO J. 6:11-16, 1987; Strittmatter and Chua, Proc. Nat. Acad. Sci. USA 84:8986-8990, 1987; Poulsen and Chua, Mol. Gen. Genet. 214:16-23, 1988; Comai et al., Plant Mol. Biol. 15:373-381, 1991; 1987; Aryan et al., Mol. Gen. Genet. 225:65-71, 1991).

Cis elements can be identified by a number of techniques, including deletion analysis, i.e., deleting one or more nucleotides from the 5′ end or internal to a promoter; DNA binding protein analysis using DNase I footprinting; methylation interference; electrophoresis mobility-shift assays, in vivo genomic footprinting by ligation-mediated PCR; and other conventional assays; or by sequence similarity with known cis element motifs by conventional sequence comparison methods. The fine structure of a cis element can be further studied by mutagenesis (or substitution) of one or more nucleotides or by other conventional methods (see for example, Methods in Plant Biochemistry and Molecular Biology, Dashek, ed., CRC Press, 1997, pp. 397-422; and Methods in Plant Molecular Biology, Maliga et al., eds., Cold Spring Harbor Press, 1995, pp. 233-300).

Cis elements can be obtained by chemical synthesis or by cloning from promoters that include such elements, and they can be synthesized with additional flanking sequences that contain useful restriction enzyme sites to facilitate subsequent manipulation. Promoter fragments may also comprise other regulatory elements such as enhancer domains, which may further be useful for constructing chimeric molecules.

Methods for construction of chimeric and variant promoters of the present invention include, but are not limited to, combining control elements of different promoters or duplicating portions or regions of a promoter (see for example, U.S. Pat. No. 4,990,607USA U.S. Pat. Nos. 4,990,607; 5,110,732USA U.S. Pat. Nos. 5,110,732; and 5,097,025USA U.S. Pat. No. 5,097,025). Those of skill in the art are familiar with the standard resource materials that describe specific conditions and procedures for the construction, manipulation, and isolation of macromolecules (e.g., polynucleotide molecules and plasmids), as well as the generation of recombinant organisms and the screening and isolation of polynucleotide molecules.

In an embodiment of the present invention, the promoter disclosed herein can be modified. Those skilled in the art can create promoters that have variations in the polynucleotide sequence. The polynucleotide sequence of the promoter of the present invention as shown in SEQ ID NO: 3 may be modified or altered to enhance their control characteristics. As one of ordinary skill in the art will appreciate, modification or alteration of the promoter sequence can also be made without substantially affecting the promoter function. The methods are well known to those of skill in the art. Sequences can be modified, for example by insertion, deletion, or replacement of template sequences in a PCR-based DNA modification approach.

The present invention encompasses functional fragments and variants of the promoter sequence disclosed herein.

A “functional fragment” herein is defined as any subset of contiguous nucleotides of the promoter sequence disclosed herein, that can perform the same, or substantially similar function as the full length promoter sequence disclosed herein. A “functional fragment” with substantially similar function to the full length promoter disclosed herein refers to a functional fragment that retains largely the same level of activity as the full length promoter sequence and exhibits the same pattern of expression as the full length promoter sequence. A “functional fragment” of the promoter sequence disclosed herein exhibits constitutive expression.

A “variant”, as used herein, is the sequence of the promoter or the sequence of a functional fragment of a promoter containing changes in which one or more nucleotides of the original sequence is deleted, added, and/or substituted, while substantially maintaining promoter function. One or more base pairs can be inserted, deleted, or substituted internally to a promoter. In the case of a promoter fragment, variant promoters can include changes affecting the transcription of a minimal promoter to which it is operably linked. Variant promoters can be produced, for example, by standard DNA mutagenesis techniques or by chemically synthesizing the variant promoter or a portion thereof.

Substitutions, deletions, insertions or any combination thereof can be combined to produce a final construct.

“Enhancer sequences” refer to the sequences that can increase gene expression. These sequences can be located upstream, within introns or downstream of the transcribed region. The transcribed region is comprised of the exons and the intervening introns, from the promoter to the transcription termination region. The enhancement of gene expression can be through various mechanisms which include, but are not limited to, increasing transcriptional efficiency, stabilization of mature mRNA and translational enhancement.

An “intron” is an intervening sequence in a gene that is transcribed into RNA and then excised in the process of generating the mature mRNA. The term is also used for the excised RNA sequences. An “exon” is a portion of the sequence of a gene that is transcribed and is found in the mature messenger RNA derived from the gene, and is not necessarily a part of the sequence that encodes the final gene product.

Many genes exhibit enhanced expression on inclusion of an intron in the transcribed region, especially when the intron is present within the first 1 kb of the transcription start site. The increase in gene expression by presence of an intron can be at both the mRNA (transcript abundance) and protein levels. The mechanism of this Intron Mediated Enhancement (IME) in plants is not very well known (Rose et al., Plant Cell, 20: 543-551 (2008) Le-Hir et al, Trends Biochem Sci. 28: 215-220 (2003), Buchman and Berg, Mol. Cell Biol. (1988) 8:4395-4405; Callis et al., Genes Dev. 1 (1987):1183-1200).

An “enhancing intron” is an intronic sequence present within the transcribed region of a gene which is capable of enhancing expression of the gene when compared to an intronless version of an otherwise identical gene. An enhancing intronic sequence might also be able to act as an enhancer when located outside the transcribed region of a gene, and can act as a regulator of gene expression independent of position or orientation (Chan et. al. (1999) Proc. Natl. Acad. Sci. 96: 4627-4632; Flodby et al. (2007) Biochem. Biophys. Res. Commun. 356: 26-31).

An intron sequence can be added to the 5′ untranslated region, the protein-coding region or the 3′ untranslated region to increase the amount of the mature message that accumulates in the cytosol.

The intron sequences can be operably linked to a promoter and a gene of interest.

The tissue expression patterns of the genes can be determined using the RNA profile database of the Massively Parallel Signature Sequencing (MPSS™). This proprietary database contains deep RNA profiles of more than 250 libraries and from a broad set of tissue types. The MPSS™ transcript profiling technology is a quantitative expression analysis that typically involves 1-2 million transcripts per cDNA library (Brenner S. et al., (2000). Nat Biotechnol 18: 630-634, Brenner S. et al. (2000) Proc Natl Acad Sci USA 97: 1665-1670). It produces a 17-base high quality usually gene-specific sequence tag usually captured from the 3′-most DpnII restriction site in the transcript for each expressed gene. The use of this MPSS data including statistical analyses, replications, etc, has been described previously (Guo M et al. (2008) Plant Mol Biol 66: 551-563).

The present invention includes a polynucleotide comprising: (i) a nucleic acid sequence of at least 90%, 91%, 92%, 93%, 94%, 95%, 96%, 97%, 98%, 99%, or 100% sequence identity, based on the Clustal V method of alignment with default parameters of KTUPLE=2, GAP PENALTY=5, WINDOW=4 and DIAGONALS SAVED=4, when compared to SEQ ID NO: 3; or (ii) a nucleic acid sequence of at least 90%, 91%, 92%, 93%, 94%, 95%, 96%, 97%, 98%, 99% or 100% sequence identity, based on the Clustal V method of alignment with default parameters of KTUPLE=2, GAP PENALTY=5, WINDOW=4 and DIAGONALS SAVED=4, when compared to a functional fragment of SEQ ID NO: 3; or (iii) a full complement of the nucleic acid sequence of (i) or (ii), wherein the polynucleotide acts as a regulator of gene expression in a plant cell.

The present invention includes a polynucleotide comprising: (i) a nucleic acid sequence of at least 90%, 91%, 92%, 93%, 94%, 95%, 96%, 97%, 98%, 99%, or 100% sequence identity, based on the Clustal V method of alignment with default parameters of KTUPLE=2, GAP PENALTY=5, WINDOW=4 and DIAGONALS SAVED=4, when compared to SEQ ID NO: 6; or (ii) a full complement of the nucleic acid sequence of (i), wherein the polynucleotide acts as a regulator of gene expression in a plant cell.

Embodiments of the Present Invention Include the Following:

One embodiment of this invention is a recombinant DNA construct comprising a promoter functional in a plant cell operably linked to an isolated polynucleotide wherein the promoter comprises a nucleic acid sequence selected from the group consisting of: (a) the nucleic acid sequence of SEQ ID NO: 3, (b) a nucleic acid sequence with at least 95% sequence identity to the nucleic acid sequence of SEQ ID NO: 3, and (c) a nucleic acid sequence comprising a functional fragment of (a) or (b). In a related embodiment, the promoter is a constitutive promoter.

In another embodiment, the recombinant construct may comprise an intron operably linked to both a promoter and an isolated polynucleotide, wherein the intron comprises a nucleic acid sequence with at least 95% identity to the nucleic acid sequence of SEQ ID NO: 6. The intron may further comprise the nucleic acid sequence of SEQ ID NO: 6. Furthermore, the expression of the isolated polynucleotide is enhanced, when compared to a control recombinant DNA construct comprising the promoter operably linked to the isolated polynucleotide, wherein neither are operably linked to the intron.

Another embodiment of this invention is a method for modulating expression of an isolated polynucleotide in a plant comprising the steps of: (a) introducing into a regenerable plant cell a recombinant DNA construct comprising a promoter functional in a plant cell operably linked to an isolated polynucleotide wherein the promoter comprises a nucleic acid sequence selected from the group consisting of: (i) the nucleic acid sequence of SEQ ID NO: 3, (ii) a nucleic acid sequence with at least 95% sequence identity to the nucleic acid sequence of SEQ ID NO: 3, and (iii) a nucleic acid sequence comprising a functional fragment of (i) or (ii); (b) regenerating a transgenic plant from the regenerable plant cell after step (a) wherein the transgenic plant comprises the recombinant DNA construct; and (c) obtaining a transgenic plant from step (b), or a progeny plant derived from the transgenic plant of step (b), wherein said transgenic plant or progeny plant comprises in its genome the recombinant DNA construct and exhibits expression of the polynucleotide.

Another embodiment of this invention is a method for modulating expression of an isolated polynucleotide in a plant comprising the steps of: (a) introducing into a regenerable plant cell a recombinant DNA construct comprising an intron sequence operably linked to both a promoter and an isolated polynucleotide wherein the intron sequence exhibits at least 95% sequence identity to SEQ ID NO: 6; (b) regenerating a transgenic plant from the regenerable plant cell after step (a) wherein the transgenic plant comprises the recombinant DNA construct; and (c) obtaining a transgenic plant from step (b), or a progeny plant derived from the transgenic plant of step (b), wherein said transgenic plant or progeny plant comprises the recombinant DNA construct and exhibits enhanced transgene expression when compared to a plant comprising a control recombinant DNA construct comprising the promoter operably linked to the isolated polynucleotide, wherein neither are operably linked to the intron.

Another embodiment of this invention is a method for modulating expression of an isolated polynucleotide in a plant comprising the steps of: (a) introducing into a regenerable plant cell a recombinant DNA construct comprising a promoter functional in a plant cell operably linked to both an intron and an isolated polynucleotide, wherein the promoter comprises the nucleic acid sequence of SEQ ID NO: 3, and wherein the intron comprises the nucleic acid sequence of SEQ ID NO: 6; (b) regenerating a transgenic plant from the regenerable plant cell after step (a) wherein the transgenic plant comprises the recombinant DNA construct; and (c) obtaining a transgenic plant from step (b), or a progeny plant derived from the transgenic plant of step (b), wherein said transgenic plant or progeny plant comprises the recombinant DNA construct and exhibits expression of the polynucleotide.

Another embodiment of this invention is a method for modulating expression of an isolated polynucleotide in a plant comprising the steps of: (a) introducing into a regenerable plant cell a recombinant DNA construct comprising a functional fragment of SEQ ID NO: 3 operably linked to an isolated polynucleotide, wherein the functional fragment comprises a nucleotide sequence selected from the group consisting of: (i) the nucleic acid sequence of SEQ ID NOS: 12, 13, 17, 21 or 25; and (ii) a nucleic acid sequence with at least 95% sequence identity to the nucleic acid sequence of SEQ ID NOS: 12, 13, 17, 21 or 25; (b) regenerating a transgenic plant from the regenerable plant cell after step (a) wherein the transgenic plant comprises the recombinant DNA construct; and (c) obtaining a transgenic plant from step (b), or a progeny plant derived from the transgenic plant of step (b), wherein said transgenic plant or progeny plant comprises the recombinant DNA construct and exhibits expression of the polynucleotide.

Another embodiment of this invention is any fragment of the disclosed promoter sequence that drives the expression of an operably linked polynucleotide in a host cell in the same or substantially similar manner as the disclosed promoter sequence.

Another embodiment of this invention is a functional fragment of SEQ ID NO: 3, that comprises at least 50, 100, 200, 300, 400, 500, 1000 or 1500 contiguous nucleotides from the 3′ end of the polynucleotide sequence of SEQ ID NO: 3.

Another embodiment of this invention is a functional fragment of SEQ ID NO: 3, wherein the fragment comprises 120 bp (SEQ ID NO: 12), 172 bp (SEQ ID NO: 13), 328 bp (SEQ ID NO: 17), 518 bp (SEQ ID NO: 21) or 1036 bp (SEQ ID NO: 25) of the 3′ end of SEQ ID NO: 3.

Another embodiment of this invention includes a functional fragment operably linked to an enhancer element. Examples include, but are not limited to, the CaMV 35S enhancer.

Another embodiment of this invention is a recombinant construct comprising a functional fragment of SEQ ID NO: 3 operably linked to an isolated polynucleotide, wherein the functional fragment comprises a nucleotide sequence selected from the group consisting of: (a) the nucleic acid sequence of SEQ ID NOS: 12, 13, 17, 21 or 25; and (b) a nucleic acid sequence with at least 95% sequence identity to the nucleic acid sequence of SEQ ID NOS: 12, 13, 17, 21 or 25.

In another embodiment, the invention includes a recombinant construct comprising a functional fragment of SEQ ID NO: 3 operably linked to both an isolated polynucleotide and an intron, wherein the functional fragment comprises a nucleotide sequence selected from the group consisting of: (a) the nucleic acid sequence of SEQ ID NOS: 12, 13, 17, 21 or 25; and (b) a nucleic acid sequence with at least 95% sequence identity to the nucleic acid sequence of SEQ ID NOS: 12, 13, 17, 21 or 25. Furthermore, the intron may comprise the nucleic acid sequence of SEQ ID NO: 6.

In another embodiment, the compositions and methods of the present invention can be used in dicots or monocots. In particular, the compositions and methods of the present invention can be used in monocotyledenous plants.

In another embodiment, the invention includes transformed plant cells, tissues, plants, and seeds. The invention encompasses regenerated, mature and fertile transgenic plants, transgenic seeds produced therefrom, T1 and subsequent generations. The transgenic plant cells, tissues, plants, and seeds may comprise at least one recombinant DNA construct of interest.

In one embodiment, the present invention encompasses plants and seeds obtained from the methods disclosed herein.

In another embodiment, the present invention includes a transgenic microorganism or cell comprising the recombinant DNA construct. The microorganism and cell may be eukaryotic, e.g., a yeast, insect or plant cell, or prokaryotic, e.g., a bacterial cell.

Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

In another embodiment, the present invention encompasses an isolated polynucleotide that functions as a promoter in a plant, wherein the polynucleotide has a nucleotide sequence that can hybridize under stringent conditions with the nucleotide sequence of SEQ ID NO: 3. The polynucleotide also may function as a constitutive promoter in a plant. The polynucleotide also may comprise at least 50, 100, 200, 300, 400, 500, 1000 or 1500 nucleotides in length. The polynucleotide also may have at least 90%, 91%, 92%, 93%, 94%, 95%, 96%, 97%, 98%, 99%, or 100% sequence identity, based on the Clustal V method of alignment with default parameters of KTUPLE=2, GAP PENALTY=5, WINDOW=4 and DIAGONALS SAVED=4, when compared to SEQ ID NO: 3.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

In another embodiment, the present invention encompasses an isolated polynucleotide that functions as a promoter in a plant and comprises a nucleotide sequence that is derived from SEQ ID NO: 3 by alteration of one or more nucleotides by at least one method selected from the group consisting of: deletion, substitution, addition and insertion. The polynucleotide also may function as a constitutive promoter in a plant. The polynucleotide also may comprise at least 50, 100, 200, 300, 400, 500, 1000 or 1500 nucleotides in length. The polynucleotide also may have at least 90%, 91%, 92%, 93%, 94%, 95%, 96%, 97%, 98%, 99%, or 100% sequence identity, based on the Clustal V method of alignment with default parameters of KTUPLE=2, GAP PENALTY=5, WINDOW=4 and DIAGONALS SAVED=4, when compared to SEQ ID NO: 3.

In another embodiment, the present invention encompasses an isolated polynucleotide comprising a nucleotide sequence, wherein the nucleotide sequence corresponds to an allele of SEQ ID NO: 3.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

### EXAMPLES

The present invention is further illustrated in the following Examples, in which parts and percentages are by weight and degrees are Celsius, unless otherwise stated. It should be understood that these examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Furthermore, various modifications of the invention in addition to those shown and described herein will be apparent to those skilled in the art from the foregoing description. Such modifications are also intended to fall within the scope of the appended claims.

### Example 1

### Description of Constitutive Promoter Selection Via MPSS Samples

Promoter candidates were identified using a set of 241 proprietary expression profiling experiments run on the MPSS (Massively Parallel Signature Sequencing) technology platform provided by Lynx Therapeutics. The 241 samples from corn consisted of various tissue samples spanning most of the range of corn tissues and developmental stages. Each experiment resulted in approximately 20,000 unique sequence tags of 17 bp length from a single tissue sample. Typically these tags could be matched to one or a few transcript sequences from the proprietary “Unicorn” EST assembly set. A query of the MPSS database was performed looking for tags that were observed in 240 or more of the 241 samples. We identified 111 tags that met the criteria and chose 22 that were observed at an expression level of 1 or greater PPM (Parts Per Million tags) in all 241 experiments for further development. Of these 22 tags. 21 mapped to a single gene based on the transcript set.

We took one of the top candidates from this list that showed strong expression across several tissue types, and identified a region of about 1500 bp containing the promoter and the first intron, defined as the first intron in the transcript from the 5′ end. These regulatory elements were designated as the P72 promoter and the P72 intron.

### Example 2

### Promoter and Intron Amplification and Cloning

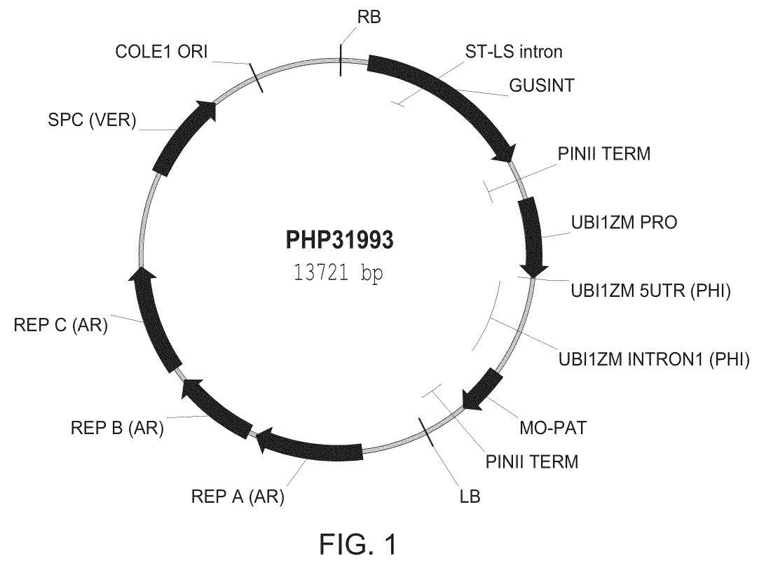

Zea mays B73 seeds were germinated in Petri plates and genomic DNA was isolated from seedling leaf tissue using the QIAGEN® DNEASY® Plant Maxi Kit (QIAGEN® Inc.) according to the manufacturer's instructions. DNA products were amplified with primers shown in Table 1 using genomic DNA as template with PHUSION® DNA polymerase (New England Biolabs Inc.). The resulting DNA fragment was cloned into the promoter testing vector PHP31993 (FIG. 1; SEQ ID NO: 2) between the AscI-NcoI restriction sites, using standard molecular biology techniques (Sambrook et al.,) or using In-fusion™ cloning from (Clontech Inc.) and sequenced completely. The expression vector containing the P72 promoter and intron was called PHP39158. The maize ubi promoter along with its 5′ UTR intron (SEQ ID NO: 1) was also cloned in the same vector and used for comparison of GUS reporter expression levels. The expression vector containing the maize ubi promoter and intron was called PHP38694. The maize ubi promoter and intron is known to confer high level constitutive expression in monocot plants (Christensen, A. H., Sharrock, R. A. and Quail, P. H., Plant Mol. Biol. 18, 675-89, 1992).

TABLE 1
Primers Used for Amplifying P72 Promoter and Intron

| | Length | | |
|---|---|---|---|
| Sequence | (nucleotides) | Forward primer | Reverse primer |
| P72 promoter with | 3938 | SEQ ID NO: 10 | SEQ ID NO: 11 |
| intron (SEQ ID | | | |
| NO: 9) | | | |

All the constructs were mobilized into the Agrobacterium strain LBA4404/pSB1 and selected on Spectinomycin and Tetracycline. Agrobacterium transformants were isolated and the integrity of the plasmid was confirmed by retransforming to E. coli or PCR analysis.

### Example 3

### Transient Assay for Promoter and Intron Activity in Maize Embryos

### Preparation of Agrobacterium Suspension

Agrobacterium was streaked from a −80° C. frozen aliquot onto a plate containing PHI-L medium and was cultured at 28° C. in the dark for 3 days. The PHI-L media comprises 890 ml H2O and Agar (HIMEDIA-CR301) 9 g/l; 50 ml/l Stock Solution A [K2HPO4 (Sigma-P2222) 60 g/l; NaH2PO4 (Sigma-S8282) 20 g/l; pH adjusted to 7.0 with KOH (HIMEDIA-RM1015)]; 50 ml/l Stock Solution B [NH4Cl (HIMEDIA-RM 730) 20 g/l; MgSO4.7H2O (HIMEDIA-RM683) 6 g/l; KCl (HIMEDIA-RM683) 3 g/l; CaCl2 (Sigma-5080) 0.2 g/l; FeSO4.7H2O (Sigma-F8048) 50 mg/l]; 10 ml/l Stock Solution C [Glucose (Sigma-G8270) 0.5 g/l and filter sterilized)]; Tetracycline (Sigma-T3383) 5 mg/l and Spectinomycin (Sigma-56501) 50 mg/l. Stock solutions A, B and C and antibiotics were added post-sterilization. The plate can be stored at 4° C. and used usually for about 1 month.

A single colony was picked from the master plate and was streaked onto a plate containing PHI-M medium [Yeast Extract (BD Difco-212750) 5 g/l; Peptone (BD Difco-211677) 10 g/l; NaCl (HIMEDIA-RM031) 5 g/l; Agar (HIMEDIA-CR301) 15 g/l; pH adjusted to 6.8 with KOH (HIMEDIA-RM1015); supplemented with Tetracycline (Sigma-T3383) 5 mg/l and Spectinomycin (Sigma-56501) 50 mg/l and incubated overnight at 28° C. in the dark.

Five ml of PHI-A media [CHU(N6) Basal salts (Sigma C-1416) 4 g/l; Erikson's vitamin solution (1000×, PhytoTechnology-E330) 1 ml/l; Thiamine.HCl (Sigma-T4625) 0.5 mg/l; 2,4-Dichloro phenoxyacetic acid (Sigma-D7299) 1.5 mg/l; L-Proline (PhytoTechnology-P698) 0.69 g/l; Sucrose (Sigma-S5390) 68.5 g/l; Glucose (Sigma-G8270) 36 g/l; pH adjusted to 5.2 with KOH (HIMEDIA-RM1015)] was added to a 14 ml falcon tube in a hood. About 3 full loops (5 mm loop size) Agrobacterium was collected from the streaked plate and suspended in the tube by vortexing. The suspension was adjusted to 0.35 Absorbance at 550 nm. The final Agrobacterium suspension was aliquoted into 2 ml microcentrifuge tubes, each containing 1 ml of the suspension. The suspension was then used as soon as possible.

### Embryo Isolation, Infection and Co-Cultivation

Immature embryos isolated from a sterilized maize ear with a sterile spatula were dropped directly into 2 ml of PHI-A medium in a microcentrifuge tube. Embryos, between 1.3 to 1.9 mm in size, were used in the experiment. The entire medium was drawn off and 1 ml of Agrobacterium suspension was added to the embryos and the tube was vortexed for 30 sec. After a 5 minute incubation, the suspension of Agrobacterium and embryos was poured into a Petri plate containing co-cultivation medium PHI-B [MS Basal salts (PhytoTechnology-M524) 4.3 g/l; B5 vitamin 1 ml from 1000× stock {Nicotinic acid (Sigma-G7126) 1 g/l, Pyridoxine HCl (Sigma-P9755) 1 g/l, Thiamine HCl (Sigma-T4625) 10 g/l)}; Myo-inositol (Sigma-13011) 0.1 g/l; Thiamine HCl (Sigma-T4625) 0.5 mg/l; 2,4-Dichloro phenoxyacetic acid (Sigma-D7299) 1 mg/l; L-Proline (PhytoTechnology-P698) 0.69 g/l; Casein acid hydrolysate vitamin free (Sigma-C7970) 0.3 g/l; Sucrose (Sigma-S5390) 30 g/l; GELRITE® (Sigma-G1910) 3 g/l; pH adjusted to 5.2 with KOH (HIMEDIA-RM1015); Post-sterilization, Silver Nitrate (Sigma-57276) 0.85 mg/l and Acetosyringone (Sigma-D134406), 1 ml from 100 mM stock, were added after cooling the medium to 45° C.]. Any embryos left in the tube were transferred to the plate using a sterile spatula. The Agrobacterium suspension was drawn off and the embryos were placed axis side down on the media. The plate was sealed with PARAFILM® and was incubated in the dark at 21° C. for 3 days.

### Resting of Co-Cultivated Embryos

For the resting step, all the embryos were transferred to a new plate containing PHI-C medium [MS Basal salts (PhytoTechnology-M524) 4.3 g/l; B5 vitamin 1 ml from 1000× stock {Nicotinic acid (Sigma-G7126) 1 g/l, Pyridoxine HCl (Sigma-P9755) 1 g/l, Thiamine HCl (Sigma-T4625) 10 g/l)}; Myo-inositol (Sigma-13011) 0.1 g/l; Thiamine HCl (Sigma-T4625) 0.5 mg/l; 2,4-Dichloro phenoxyacetic acid (Sigma-D7299) 2 mg/l; L-Proline (PhytoTechnology-P698) 0.69 g/l; Casein acid hydrolysate vitamin free (Sigma-C7970) 0.3 g/l; Sucrose (Sigma-S5390) 30 g/l; MES buffer (Fluka-69892) 0.5 g/l; GELRITE® (Sigma-G1910) 3 g/l; pH was adjusted to 5.8 with KOH (HIMEDIA-RM1015); Post-sterilization, Silver Nitrate (Sigma-57276) 0.85 mg/l and Carbenicillin (Sigma-C3416) 0.1 g/l were added after cooling the medium to 45° C.]. The plates were sealed with PARAFILM® and incubated in the dark at 28° C.

### Histochemical and Fluorometric GUS Analysis

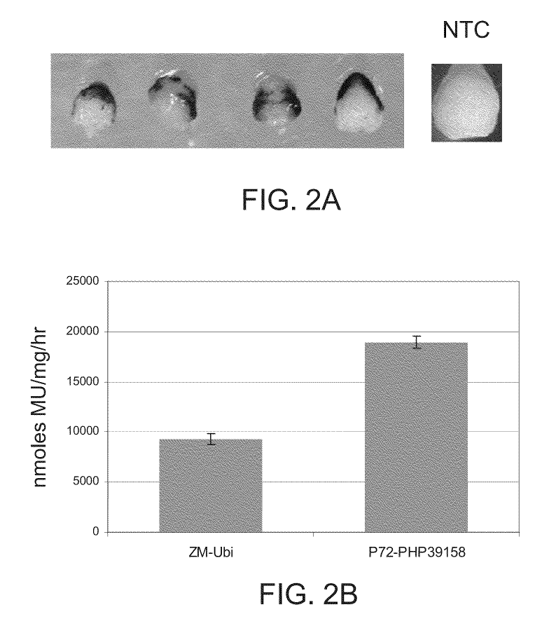

Transient GUS expression was analyzed in embryos after 3 days of resting. Ten embryos for each construct were used for histochemical GUS staining with 5-bromo-4-chloro-3-indolyl-β-D-glucuronide (X-Gluc), using standard protocols (Janssen and Gardner, Plant Mol. Biol. (1989) 14:61-72,) and two pools of 5 embryos were used for quantitative MUG assay using standard protocols (Jefferson, R. A., Nature. 342, 837-8 (1989); Jefferson, R. A., Kavanagh, T. A. & Bevan, M. W. EMBO J. 6:3901-3907 (1987). High level of GUS expression was observed in embryos infected with P72 promoter and intron construct, indicating that the P72 Promoter and Intron together are able to drive GUS reporter gene expression in maize embryos (FIGS. 2A and 2B). The GUS expression in Maize embryos infected with P72 promoter (SEQ ID NO: 3) and intron (SEQ ID NO: 6) construct was more than that observed with the Maize Ubi promoter and intron (SEQ ID NO: 1). Non-transgenic Control refers to embryos or calli that were not infected with Agrobacterium but were otherwise subjected to the identical treatment as Agrobacterium infected test samples.

### Example 4

### Transformation and Regeneration of Maize Callus Via Agrobacterium

For obtaining transgenic maize plants with stable expression of recombinant construct (PHP39158) with P72 promoter (SEQ ID NO: 3) and intron (SEQ ID NO: 6) driving reporter gene expression, and of control recombinant vector, the co-cultivated embryos obtained as described in example 3 were processed as described below.

After 12 days of resting all of the co-cultivated embryos were transferred to new plates containing PHI-D medium [PHI-C medium supplemented with Bialaphos (Gold Bio-B0178) 1.5 mg/l] for three weeks to select stable transgenic events. The Bialaphos concentration was increased to 3 mg/l for the remainder of the selection period. The plates were sealed and incubated in the dark at 28° C. The embryos were transferred to fresh selection medium at two-week intervals for a total of about 2 months. The Bialaphos-resistant calli were then “bulked” up by growing on the same medium for another two weeks until the diameter of the Calli was about 1.5-2 cm.

For maturation, the Calli were then cultured on PHI-E medium [MS salts (PhytoTechnology-M524) 4.3 g/l; MS vitamins 5 ml from 200× stock {Glycine (Sigma-7126) 0.4 g/l; Nicotinic acid (Sigma-G7126) 0.1 g/l; Pyridoxine HCl (Sigma-P9755) 0.1 g/l; Thiamine HCl (Sigma-T4625) 0.02 g/l}; Myo-inositol (Sigma-13011) 0.1 g/l; Zeatin (Sigma-Z0164) 0.5 mg/l; Sucrose (Sigma-S5390) 60.0 g/l; Ultra pure Agar-Agar (EMD-1.01613.1000) 6.0 g/l; Post sterilization, Indoleacetic acid (IAA, Sigma-15148) 0.1 mg/l; Abscisic acid (Sigma-A4906) 26 micrograms/l; Bialaphos (Gold Bio B0178) 1.5 mg/l; Carbenicillin (Sigma-C3416) 0.1 g/l were added and pH was adjusted to pH 5.6] in the dark at 28° C. for 1-3 weeks to allow somatic embryos to mature. The matured Calli were then cultured on PHI-F medium for regeneration [MS salts (PhytoTechnology-M524) 4.3 g/l; MS vitamins 5 ml from 200× stock {Glycine(Sigma-7126) 0.4 g/l; Nicotinic acid (Sigma-G7126) 0.1 g/l; Pyridoxine HCl (Sigma-P9755) 0.1 g/l; Thiamine Hcl (Sigma-T4625) 0.2 g/l}; Myo-inositol (Sigma-13011) 0.1 g/l; Sucrose (Sigma-S5390) 40.0 g/l; Agar (Sigma-A7921) 6 g/l; Bialaphos 1.5 g/l; pH 5.6] at 25° C. under a daylight schedule of 16 hours light (270 uE m−2 sec−1) and 8 hours dark until shoots and roots are developed. Each small plantlet was then transferred to a 25×150 mm tube containing PHI-F medium and grown under the same conditions for approximately another week. The plants were transplanted to soil mixture in a green house.

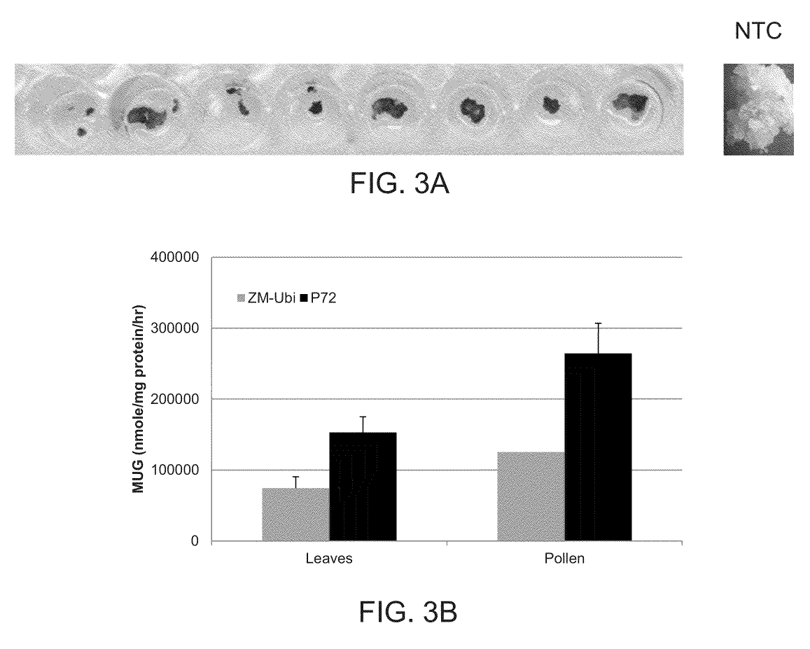

GUS reporter gene expressing plants were determined in the regenerated plants. Strong GUS reporter gene expression was observed in leaf tissue as well as pollen from all stable transgenic events generated with P72 promoter and intron recombinant construct (FIGS. 3A and 3B).

Maize plants can be transformed with recombinant constructs expressing any polynucleotide of interest, with the expression being driven by P72 promoter and intron, and transgenic plants can be obtained as described in Example 3 and Example 4.

### Example 5

### Transformation and Regeneration of Rice Callus Via Agrobacterium Infection

A single Agrobacterium colony from a freshly streaked plate was inoculated in YEB liquid media [Yeast extract (BD Difco-212750) 1 g/l; Peptone (BD Difco-211677) 5 g/l; Beef extract (Amresco-0114) 5 g/l; Sucrose (Sigma-S5390) 5 g/l; Magnesium Sulfate (Sigma-M8150) 0.3 g/l at pH-7.0] supplemented with Tetracycline (Sigma-T3383) 2.5 mg/l and Spectinomycin (Sigma-5650) 50 mg/l. The cultures were grown overnight at 28° C. in dark with continuous shaking at 220 rpm. The following day the cultures were adjusted to 0.5 Absorbance at 550 nm in PHI-A media (see Example 2 for composition of PHI-A) supplemented with 200 μM Acetosyringone (Sigma-D134406) and incubated for 1 hour at 28° C. with continuous shaking at 220 rpm.

Japonica rice var nipponbare seeds were sterilized in absolute ethanol for 10 minutes then washed 3 times with water and incubated in 70% Sodium hypochlorite [Fisher Scientific-27908] for 30 minutes. The seeds were then washed 5 times with water and dried completely. The dried seeds were inoculated into NB-CL media [CHU(N6) basal salts (PhytoTechnology-C416) 4 g/l; Eriksson's vitamin solution (1000× PhytoTechnology-E330) 1 ml/l; Thiamine HCl (Sigma-T4625) 0.5 mg/l; 2,4-Dichloro phenoxyacetic acid (Sigma-D7299) 2.5 mg/l; BAP (Sigma-B3408) 0.1 mg/l; L-Proline (PhytoTechnology-P698) 2.5 g/l; Casein acid hydrolysate vitamin free (Sigma-C7970) 0.3 g/l; Myo-inositol (Sigma-13011) 0.1 g/l; Sucrose (Sigma-S5390) 30 g/l; GELRITE® (Sigma-G1101.5000) 3 g/l; pH 5.8) and allowed to grow at 28° C. in light.

15-21 day old proliferating calli were transferred to a sterile culture flask and Agrobacterium solution prepared as described above was added to the flask. The suspension was incubated for 20 minutes at 25° C. with gentle shaking every 5 minutes. The Agrobacterium suspension was decanted carefully and the calli were placed on Whatman filter paper No-4. The calli were immediately transferred to NB-CC medium [NB-CL supplemented with 200 μM Acetosyrigone (Sigma-D134406) and incubated at 21° C. for 72 hrs.

### Culture Termination and Selection

The co-cultivated calli were placed in a dry, sterile, culture flask and washed with 1 liter of sterile distilled water containing Cefotaxime (Duchefa-C0111.0025) 0.250 g/l and Carbenicillin (Sigma-C0109.0025) 0.4 g/l. The washes were repeated 4 times or until the solution appeared clear. The water was decanted carefully and the calli were placed on Whatman filter paper No-4 and dried for 30 minutes at room temperature. The dried calli were transferred to NB-RS medium [NB-CL supplemented with Cefotaxime (Duchefa-C0111.0025) 0.25 g/l; and Carbenicillin (Sigma-C0109.0025) 0.4 g/l and incubated at 28° C. for 4 days.

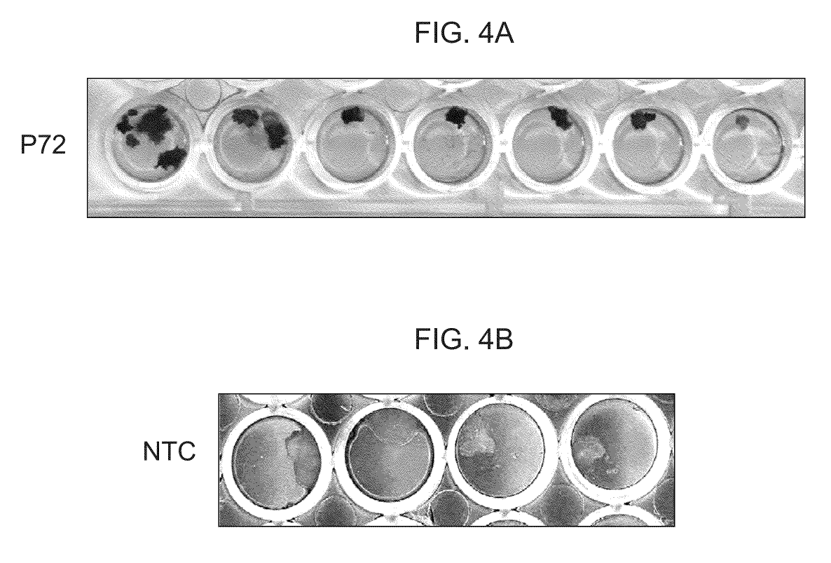

The calli were then transferred to NB-SB media [NB-RS supplemented with Bialaphos (Meiji Seika K.K., Tokyo, Japan) 5 mg/l and incubated at 28° C. and subcultured into fresh medium every 14 days. After 35-40 days on selection, proliferating, Bialaphos resistant, callus events were easily observable. A small piece from each callus event was used for histochemical GUS staining with 5-bromo-4-chloro-3-indolyl-β-D-glucuronide (X-Gluc), using standard protocols (Janssen and Gardner, Plant Mol. Biol. (1989) 14:61-72). High level of GUS reporter gene expression was observed in stable callus events obtained with P72 promoter with intron construct, indicating that the P72 Promoter (SEQ ID NO: 3) and Intron (SEQ ID NO: 6) together are able to drive GUS reporter gene expression in stable rice callus events (FIG. 4).

### Example 6

### Regeneration of Stable Rice Plants from Transformed Rice Calli

Transformed callus events obtained as described in Example 5 can be further subcultured to obtain stable transgenic plants. Remaining callus events can be transferred to NB-RG media [CHU(N6) basal salts (PhytoTechnology-C416) 4 g/l; N6 vitamins 1000×1 ml {Glycine (Sigma-47126) 2 g/l; Thiamine HCl (Sigma-T4625) 1 g/l; Pyridoxine HCl (Sigma-P9755) 0.5 g/l; Nicotinic acid (Sigma-N4126) 0.5 g/l}; Kinetin (Sigma-K0753) 0.5 mg/l; Casein acid hydrolysate vitamin free (Sigma-C7970) 0.5 g/l; Sucrose (Sigma-S5390) 20 g/l; Sorbitol (Sigma-S1876) 30 g/l, pH was adjusted to 5.8 and 4 g/l GELRITE® (Sigma-G1101.5000) was added. Post-sterilization 0.1 ml/l of CuSo4 (100 mM concentration, Sigma-C8027) and 100 ml/l AA Amino acids 100× {Glycine (Sigma-G7126) 75 mg/l; L-Aspartic acid (Sigma-A9256) 2.66 g/l; L-Arginine (Sigma-A5006) 1.74 g/l; L-Glutamine (Sigma-G3126) 8.76 g/l} and incubated at 32 C in light. After 15-20 days, regenerating plantlets can be transferred to magenta boxes containing NB-RT media [MS basal salts (PhytoTechnology-M524) 4.33 g/L; B5 vitamin 1 ml/l from 1000× stock {Nicotinic acid (Sigma-G7126) 1 g/l, Pyridoxine HCl (Sigma-P9755) 1 g/l, Thiamine HCl (Sigma-T4625) 10 g/l)}; Myo-inositol (Sigma-13011) 0.1 g/l; Sucrose (Sigma-S5390) 30 g/l; and IBA (Sigma-15386) 0.2 mg/l; pH adjusted to 5.8]. Rooted plants obtained after 10-15 days can be hardened in liquid Y media [1.25 ml each of stocks A-F and water sufficient to make 1000 ml. Composition of individual stock solutions: Stock (A) Ammonium Nitrate (HIMEDIA-RM5657) 9.14 g/l, (B) Sodium hydrogen Phosphate (HIMEDIA-58282) 4.03 g, (C) Potassium Sulphate (HIMEDIA-29658-4B), (D) Calcium Chloride (HIMEDIA-C5080) 8.86 g, (E) Magnesium Sulphate (HIMEDIA-RM683) 3.24 g, (F) (Trace elements) Magnesium chloride tetrahydrate (HIMEDIA-10149) 15 mg, Ammonium Molybdate (HIMEDIA-271974B) 6.74 mg/l, Boric acid (Sigma-136768) 9.34 g/l, Zinc sulphate heptahydrate (HiMedia-RM695) 0.35 mg/l, Copper Sulphate heptahydrate (HIMEDIA-C8027) 0.31 mg/l, Ferric chloride hexahydrate (Sigma-236489) 0.77 mg/l, Citric acid monohydrate (HIMEDIA-C4540) 0.119 g/l] at 28° C. for 10-15 days before transferring to greenhouse. Each independent event can be transferred to an individual pot and the GUS reporter gene expression can be analyzed in different tissues of the transgenic plant.

### Example 7

### Copy Number Analysis in Transgenic Maize and Rice Plants

Transgenic plants are analyzed for copy number using TaqMan-based quantitative real-time PCR (qPCR) analysis. All single copy events are transferred to individual pots and further analysis is performed only on these.

### Transgene Copy Number Determination by Quantitative PCR

Transgenic corn and rice plants generated using P72-containing PHP39158 construct were analyzed to determine the transgene copy number using TaqMan-based quantitative real-time PCR (qPCR) analysis. Genomic DNA was isolated from the leaf tissues collected from 10-day old T0 corn and rice plants using the QIAGEN® DNEASY® Plant Maxi Kit (QIAGEN® Inc.) according to the manufacturer's instructions. DNA concentration was adjusted to 100 ng/μl and was used as a template for the qPCR reaction to determine the copy number. The copy number analysis was carried out by designing PCR primers and TaqMan probes for the target gene and for the endogenous glutathione reductase 5 (GR5) and alcohol dehydrogenase (ADH) genes. The endogenous GR5 gene serves as an internal control for rice and ADH gene serves as internal control for corn to normalize the Ct values obtained for the target gene across different samples. In order to determine the relative quantification (RQ) values for the target gene, genomic DNA from known single and two copy calibrators for a given gene were also included in the experiment. Test samples and calibrators were replicated twice for accuracy. Non-transgenic control and no template control were also included in the reaction. The reaction mixture (for a 20 μl reaction volume) comprises 10 μl of 2× TaqMan universal PCR master mix (Applied Biosystems), 0.5 μl of 10 μM PCR primers and 0.5 μl of 10 μM TaqMan probe for both target gene and endogenous gene. Volume was adjusted to 19 μl using sterile Milli Q water and the reaction components were mixed properly and spun down quickly to bring the liquid to bottom of the tube. Nineteen μl of the reaction mix was added into each well of reaction plate containing 1 μl of genomic DNA to achieve a final volume of 20 μl. The plate was sealed properly using MicroAmp optical adhesive tape (Applied Biosystems) and centrifuged briefly before loading onto the Real time PCR system (7500 Real PCR system, Applied Biosystems). The amplification program used was: 1 cycle each of 50° C. for 2:00 min and 95° C. for 10:00 min followed by 40 repetitions of 95° C. for 15 sec and 58° C. for 1:00 min. After completion of the PCR reaction, the SDS v2.1 software (Applied Biosystems) was used to calculate the RQ values in the test samples with reference to single copy calibrator.

PCR primers and TaqMan probes designed for the GUS reporter gene and for the endogenous GR5 and ADH genes are listed in the following Tables.

TABLE 2
Primer Sequences

| | | SEQ |
|---|---|---|
| Primer ID | Sequence (5′ to 3′) | ID NO: |
| GUS F primer | CTTACGTGGCAAAGGATTCGA | 29 |
| GUS R primer | GCCCCAATCCAGTCCATTAA | 30 |
| GR5 F primer | GGCAGTTTGGTTGATGCTCAT | 31 |
| GR5 R primer | TGCTGTATATCTTTGCTTTGAACCAT | 32 |
| ADH F primer | CAAGTCGCGGTTTTCAATCA | 33 |
| ADH R primer | TGAAGGTGGAAGTCCCAACAA | 34 |

TABLE 3
Probe Sequences

| | SEQ ID NO: | Probe | Quencher |
|---|---|---|---|
| GUS | SEQ ID NO: 35 | Fam | Tamra |
| GR5 | SEQ ID NO: 36 | Vic | MGB |
| ADH | SEQ ID NO: 37 | Vic | MGB |

All single copy events were transferred to individual pots and further analysis was performed on different tissues collected from T0 and T1 corn and T0 rice plants.

### Example 8

### Qualitative and Quantitative Analysis of GUS Reporter Gene Expression in Stable Maize and Rice Events

Both qualitative and quantitative GUS reporter gene expression analyses were carried out in triplicates on at least 5 independent single copy events. Different tissue samples were collected for histochemical GUS staining with 5-bromo-4-chloro-3-indolyl-β-D-glucuronide (X-Gluc), using standard protocols (Janssen and Gardner, Plant Mol. Biol. (1989) 14:61-72) and for quantitative MUG assay using standard protocols (Jefferson, R. A., Nature. 342, 837-8 (1989); Jefferson, R. A., Kavanagh, T. A. & Bevan, M. W., EMBO J. 6, 3901-3907 (1987).

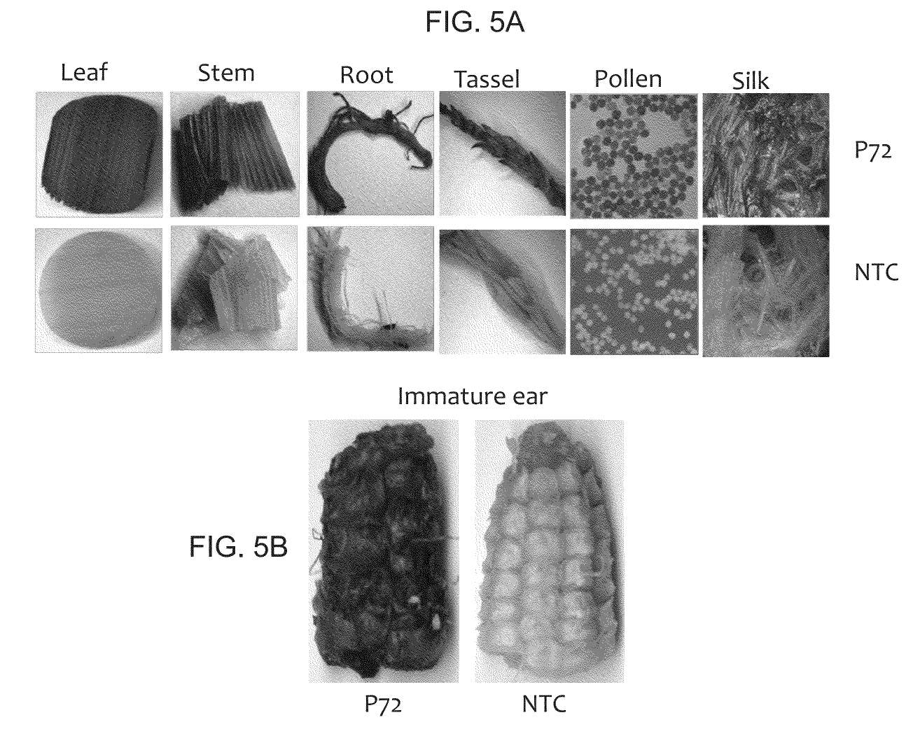

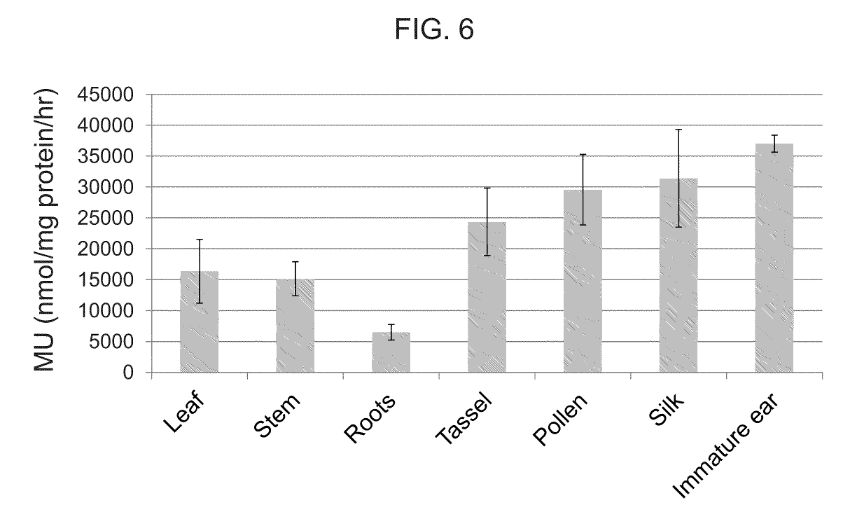

GUS reporter gene expression was determined in T1 corn plants. Strong GUS reporter gene expression was observed in leaves, stem, roots, tassel, pollen, silk and immature ear from T1 corn events (FIGS. 5A, 5B and 6).

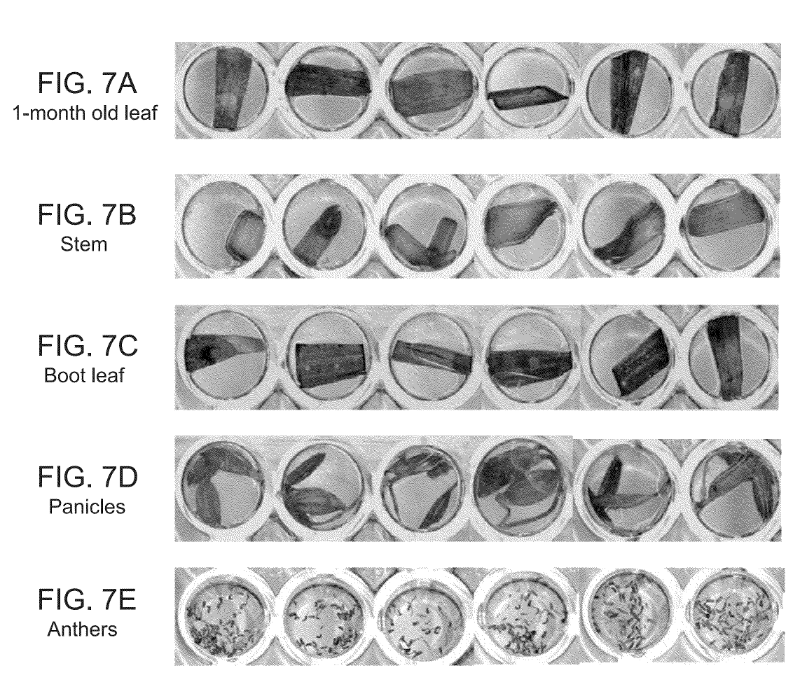

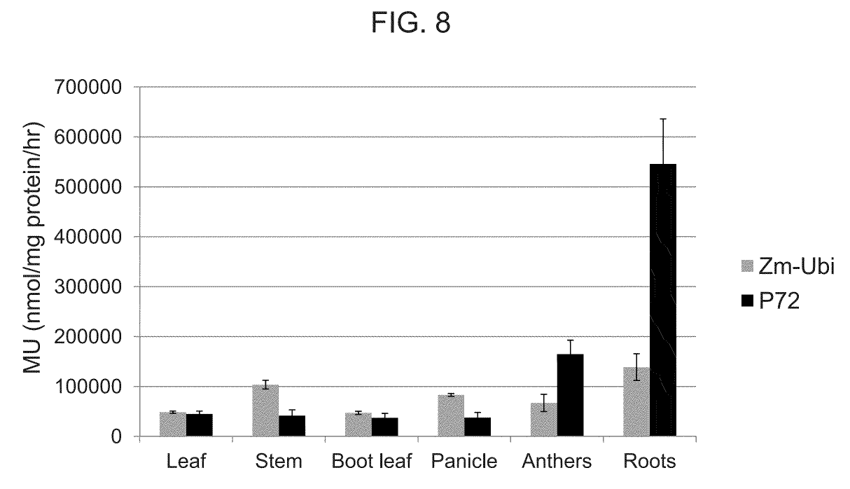

In rice, the GUS reporter gene expression measured in different tissues collected from T0 events was compared with the data collected from rice events carrying Zm-Ubi promoter and intron driving GUS expression in transgenic rice plants (FIGS. 7A-7E and 8). In anthers and roots the level of GUS reporter gene expression is significantly higher with P72 promoter and intron compared to Zm-Ubi promoter and intron (FIG. 8).

### Example 9

### Promoter Truncation Constructs and Testing of Truncated Promoter Strength

The sequence of the P72 promoter can be truncated from the 5′ end to identify the minimal sequence that can still drive high level transcription of a downstream gene. In order to test this, primers can be designed to amplify and clone different P72 promoter truncations. Intronless promoter and promoterless intron constructs can also be tested. Promoter truncations can be made with various lengths of the promoter such as 0 kb (only intron), 0.172 kb, 0.328 kb, 0.518 kb and 1.036 kb of P72 promoter sequence upstream of the intron sequence. These sequences can be amplified with PHUSION® DNA polymerase (New England Biolabs Inc.) and cloned into the promoter testing vector PHP31993 (FIG. 1) between the AscI-NcoI restriction sites, using standard molecular biology techniques (Sambrook et al.,) or using In-Fusion™ cloning from Clontech Inc.

TABLE 4
Primers Used for Cloning Different Fragments of
P72 Promoter and Intron

| | | SEQ ID NO | |
|---|---|---|---|
| | Length with P72 | Forward | Reverse |
| Amplified Fragment | Intron (nucleotides) | Primer | Primer |
| P72 intron only | 2386 | 7 | 8 |
| (SEQ ID NO: 6) | | | |
| P72₁₇₂ + P72 intron | 2558 | 15 | 16 |
| (SEQ ID NO: 14) | | | |
| P72₃₂₈ + P72 intron | 2714 | 19 | 20 |
| (SEQ ID NO: 18) | | | |
| P72₅₁₈ + P72 intron | 2904 | 23 | 24 |
| (SEQ ID NO: 22) | | | |
| P72₁₀₃₆ + P72 intron | 3422 | 27 | 28 |
| (SEQ ID NO: 26) | | | |
| P72₁₅₅₂ (no intron) | 1552 | 4 | 5 |
| (SEQ ID NO: 3) | | | |

All the resulting constructs can be mobilized into the Agrobacterium strain LBA4404/pSB1 and selected on Spectinomycin and Tetracycline as explained in Example 3. Agrobacterium transformants can be isolated and the integrity of the plasmid can be confirmed by retransforming to E. coli or PCR analysis. Stable transgenic rice plants can be generated and the activity of the different P72 truncations can be determined by analyzing the target gene expression in different tissues, as explained in Example 8.

### Example 10

### Testing of Promoter and Intron with Heterologous Elements

The strength of the P72 promoter and intron sequences in driving the expression of a target gene can be tested by cloning the P72 promoter with heterologous introns and the P72 intron with heterologous promoters. The resulting constructs can be tested in stable transgenic rice plants to check the strength of target gene expression in different tissues, as explained in Example 8.

1. A recombinant DNA construct comprising a promoter functional in a plant cell operably linked to an isolated polynucleotide, wherein the promoter comprises a nucleic acid sequence selected from the group consisting of:
(a) the nucleic acid sequence of SEQ ID NO: 3,
(b) a nucleic acid sequence with at least 95% sequence identity to the nucleic acid sequence of SEQ ID NO: 3, and
(c) a nucleic acid sequence comprising a functional fragment of (a) or (b).

2. The recombinant DNA construct of claim 1, wherein the promoter and the isolated polynucleotide are each operably linked to an intron, and further wherein the intron comprises a nucleic acid sequence with at least 95% sequence identity to the nucleic acid sequence of SEQ ID NO: 6.

3. A recombinant DNA construct comprising an intron operably linked to both a promoter and an isolated polynucleotide, wherein the intron comprises a nucleic sequence with at least 95% sequence identity to SEQ ID NO: 6.

4. The recombinant DNA construct of claim 3, wherein the intron comprises the nucleic acid sequence of SEQ ID NO: 6.

5. The recombinant DNA construct of claim 2, wherein expression of the isolated polynucleotide in plants is enhanced, when compared to a control recombinant DNA construct comprising the promoter operably linked to the isolated polynucleotide, wherein neither are operably linked to the intron.

6. The recombinant DNA construct of claim 1 wherein the promoter is a constitutive promoter.

7. A plant comprising the recombinant DNA construct of claim 1.

8. A seed comprising the recombinant DNA construct of claim 1.

9. A plant comprising the recombinant DNA construct of claim 2.

10. A seed comprising the recombinant DNA construct of claim 2.

11. A plant comprising the recombinant DNA construct of claim 3.

12. A seed comprising the recombinant DNA construct of claim 3.

13. A plant comprising the recombinant DNA construct of claim 4.

14. A seed comprising the recombinant DNA construct of claim 4.

15. A method for modulating expression of an isolated polynucleotide in a plant comprising the steps of:
(a) introducing into a regenerable plant cell the recombinant DNA construct of claim 1;
(b) regenerating a transgenic plant from the regenerable plant cell after step (a), wherein the transgenic plant comprises the recombinant DNA construct; and
(c) obtaining a progeny plant derived from the transgenic plant of step (b), wherein said progeny plant comprises the recombinant DNA construct and exhibits expression of the polynucleotide.

16. A method for modulating expression of an isolated polynucleotide in a plant comprising the steps of:
(a) introducing into a regenerable plant cell the recombinant DNA construct of claim 3, wherein the promoter is functional in a plant cell;
(b) regenerating a transgenic plant from the regenerable plant cell after step (a) wherein the transgenic plant comprises the recombinant DNA construct; and
(c) obtaining a progeny plant derived from the transgenic plant of step (b), wherein said progeny plant comprises the recombinant DNA construct and exhibits enhanced expression of the isolated polynucleotide when compared to a plant comprising a control recombinant DNA construct comprising the promoter operably linked to the isolated polynucleotide, wherein neither are operably linked to the intron.

17. The method of claim 15, wherein the
promoter functional in a plant cell is operably linked to both an intron and an isolated polynucleotide, wherein the promoter comprises the nucleic acid sequence of SEQ ID NO: 3, and wherein the intron comprises the nucleic acid sequence of SEQ ID NO: 6.

18. The method of claim 15, wherein said plant is a monocot.

19. The method of claim 16, wherein said plant is a monocot.

20. The method of claim 17, wherein said plant is a monocot.

21. The recombinant DNA construct of claim 3, wherein expression of the isolated polynucleotide in plants is enhanced, when compared to a control recombinant DNA construct comprising the promoter operably linked to the isolated polynucleotide, wherein neither are operably linked to the intron.

22. The recombinant DNA construct of claim 4, wherein expression of the isolated polynucleotide in plants is enhanced, when compared to a control recombinant DNA construct comprising the promoter operably linked to the isolated polynucleotide, wherein neither are operably linked to the intron.
